(19) United States
(12) Patent Application Publication
(10) Pub. No.: US 2014/0141037 A1
(43) Pub. Date: May 22, 2014

(54) RSV F PREFUSION TRIMERS

(71) Applicants: Kurt Swanson, Newton, MA (US); Andrea Carfi, Cambridge, MA (US)

(72) Inventors: Kurt Swanson, Newton, MA (US); Andrea Carfi, Cambridge, MA (US)

(73) Assignee: Novartis AG, Basel (CH)

(21) Appl. No.: 14/084,494

(22) Filed: Nov. 19, 2013

Related U.S. Application Data
(60) Provisional application No. 61/728,498, filed on Nov. 20, 2012.

(51) Int. Cl. C07K 14/005

(52) U.S. Cl. CPC C07K 14/005
USPC 424/196.11; 530/350; 435/70.3; 435/68.1; 435/70.1

(57) ABSTRACT

Complexes that contain RSV F ectodomain polypeptides and methods for making the complexes are disclosed. The RSV F ectodomain polypeptides can be in the prefusion form.

### RELATED APPLICATIONS

This application claims the benefit of U.S. Patent Application No. 61/728,498, filed on Nov. 20, 2012. The entire teaching of the above application is incorporated herein by reference.

### SEQUENCE LISTING

The instant application contains a Sequence Listing which has been submitted electronically in ASCII format and is hereby incorporated by reference in its entirety. Said ASCII copy, created on Nov. 18, 2013, is named PAT055275-US-NP_SL.txt and is 75,311 bytes in size.

### BACKGROUND OF THE INVENTION

Respiratory syncytial virus (RSV) is an enveloped non-segmented negative-strand RNA virus in the family Paramyxoviridae, genus Pneumovirus. It is the most common cause of bronchiolitis and pneumonia among children in their first year of life. RSV also causes repeated infections including severe lower respiratory tract disease, which may occur at any age, especially among the elderly or those with compromised cardiac, pulmonary, or immune systems.

To infect a host cell, paramyxoviruses such as RSV, like other enveloped viruses such as influenza virus and HIV, require fusion of the viral membrane with a host cell's membrane. For RSV the conserved fusion protein (RSV F) fuses the viral and cellular membranes by coupling irreversible protein refolding with juxtaposition of the membranes. In current models based on paramyxovirus studies, the RSV F protein initially folds into a metastable “pre-fusion” conformation. During cell entry, the pre-fusion conformation undergoes refolding and conformational changes to its stable “post-fusion” conformation.

The RSV F protein is translated from mRNA into an approximately 574 amino acid protein designated F₀. Post-translational processing of F₀ includes removal of an N-terminal signal peptide by a signal peptidase in the endoplasmic reticulum. F₀ is also cleaved at two sites (approximately 109/110 and approximately 136/137) by cellular proteases (in particular furin) in the trans-Golgi. This cleavage results in the removal of a short intervening sequence and generates two subunits designated F₁ (˜50 kDa; C-terminal; approximately residues 137-574) and F₂ (˜20 kDa; N-terminal; approximately residues 1-109) that remain associated with each other. F₁ contains a hydrophobic fusion peptide at its N-terminus and also two amphipathic heptad-repeat regions (HRA and HRB). HRA is near the fusion peptide and HRB is near the transmembrane domain. Three F₁-F₂ heterodimers are assembled as homotrimers of F₁-F₂ in the virion.

A vaccine against RSV infection is not currently available but is desired. One potential approach to producing a vaccine is a subunit vaccine based on purified RSV F protein. However, for this approach it is desirable that the purified RSV F protein is in a single form and conformation that is stable over time, consistent between vaccine lots, and conveniently purified.

The RSV F protein can be truncated, for example by deletion of the transmembrane domain and cytoplasmic tail, to permit its expression as an ectodomain, which may be soluble. In addition, although RSV F protein is initially translated as a monomer, the monomers are cleaved and assemble into trimers. When RSV F protein is in the form of cleaved trimers, the hydrophobic fusion peptide is exposed. The exposed hydrophobic fusion peptides on different trimers, e.g., soluble ecto-domain trimers, can associate with each other, resulting in the formation of rosettes. The hydrophobic fusion peptides can also associate with lipids and lipoproteins, for example from cells that are used to express recombinant soluble RSV F protein. Due to the complexity of RSV F protein processing, structure and refolding, purified, homogeneous, immunogenic preparations are difficult to obtain.

The pre-fusion form of RSV F contains epitopes that are not present on the post-fusion form. See, e.g., Magro, M. et al., Proc. Natl. Acad. Sci. USA, 109(8):3089-94 (2012)). Thus, for vaccines, the stabilized pre-fusion form is generally considered more desirable antigenically. Several RSV F constructs have been generated using the general theme of GCN-stabilization. However, in each case, whether the HRB was stabilized with a GCN, engineered disulfide bonds or point mutations designed to strengthen the trimer HRB hydrophobic core interactions, the result was a protein that was not expressed and exported from the cell efficiently. Attempts to make a post-fusion RSV F that has mutations to its furin cleavage sites to prevent fusion peptide release resulted in failure of the RSV F to form trimers similar to those observed in the well studied parainfluenza virus F's.

Thus, there is a need for improved RSV F protein compositions and methods for making RSV F protein compositions.

### SUMMARY OF THE INVENTION

The invention relates to respiratory syncytial virus F (RSV F) complexes that comprise three RSV F ectodomain polypeptides, each comprising an endogenous HRA region, and at least one oligomerization polypeptide, wherein the three ectodomain polypeptides and the at least one oligomerization polypeptide form a six-helix bundle, provided that the endogenous HRA regions of the RSV F polypeptides are not part of the six-helix bundle. Optionally, each RSV F ectodomain polypeptide may comprise an HRB region and each oligomerization polypeptide may comprise an oligomerization region. The six helix bundle can comprise the HRB region of each RSV F ectodomain and the oligomerization region of each oligomerization peptide. The oligomerization region can comprise an RSV F HRA amino acid sequence. Optionally, the complex can consist of the three RSV F ectodomain polypeptides and three oligomerization polypeptides. One or more of the oligomerization polypeptides can further comprise a functional region that is operably linked to the oligomerization region. The functional regions can be independently selected from the group consisting of an immunogenic carrier protein, an antigen, a particle-forming polypeptide, a lipid, and polypeptides that can associate the oligomerization polypeptide with a liposome or particle. The functional region can be an antigen. The antigen can be RSV G. Optionally, one or more of the RSV F ectodomain polypeptides is an uncleaved RSV F ectodomain polypeptide. Optionally, one or more of the RSV F ectodomain polypeptides is a cleaved RSV F ectodomain polypeptide. Optionally, each of the RSV F ectodomain polypeptides contains one or more altered furin cleavage sites. The amino acid sequence of the RSV F ectodomain polypeptides can be selected from the group consisting of: SEQ ID NO: 8 (Del21 Furx), SEQ ID NO: 3 (Furmt), SEQ ID NO: 4 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag, is omitted. At least one of the RSV F ectodomain polypeptide can be a recombinant polypeptide that comprises a C-terminal 6-helix bundle forming moiety. The C-terminal six-helix bundle forming moiety can comprise a heptad repeat region of the fusion protein of an enveloped virus. The heptad repeat region can be the HRA or HRB from a Type I fusion protein of an enveloped virus. For example, the heptad repeat region can be selected from the group consisting of RSV F HRA, RSV F HRB, and HIV gp41 HRA. Optionally, the six-helix bundle comprises the C-terminal 6-helix bundle forming moiety of three recombinant RSV F ectodomain polypeptides and the oligomerization region of each oligomerization peptide. The RSV F ectodomain polypeptides can be in the pre-fusion conformation. The RSV F complex can be characterized by a rounded shape when viewed in negatively stained electron micrographs. The RSV F complex can comprise prefusion epitopes that are not present on post-fusion forms of RSV F.

The invention also relates to a respiratory syncytial virus F (RSV F) complex, that comprises three RSV F ectodomain polypeptides that each contains an endogenous HRA region and an endogenous HRB region, at least one of the RSV F ectodomain polypeptides further comprise a C-terminal 6-helix bundle forming moiety, wherein the complex is characterized by a six-helix bundle formed by the C-terminal 6-helix bundle forming moiety and the endogenous HRB region.

The invention also relates to a method for producing a respiratory syncytial virus F (RSV F) complex, that comprises (a) providing RSV F protein ectodomain polypeptides and at least one oligomerization polypeptide, and (b) combining the RSV F ectodomain polypeptides and the at least one oligomerization polypeptide under conditions suitable for the formation of a RSV F complex, whereby a RSV F complex is produced in which three of said RSV F ectodomain polypeptides and at least one of said oligomerization polypeptides form a six-helix bundle, provided that the endogenous HRA regions of the RSV F ectodomain polypeptides are not part of the six-helix bundle. The RSV F ectodomain polypeptides provided in (a) can be uncleaved RSV F ectodomain polypeptides. The RSV F ectodomain polypeptides provided in (a) can contain one or more altered furin cleavage sites. The RSV F ectodomain polypeptides provided in (a) can be purified monomers. Optionally, the method can further comprise (c) cleaving the RSV F protein ectodomain polypeptides in the produced complex with a protease. The RSV F protein ectodomain polypeptides provided in (a) can be expressed in insect cells, mammalian cells, avian cells, yeast cells, Tetrahymena cells, or combinations thereof. Each RSV F ectodomain polypeptide can comprise an HRB region and each exogenous oligomerization polypeptide can comprise an oligomerization region. The six-helix bundle can comprise the HRB region of each RSV F ectodomain polypeptide and the oligomerization region of each oligomerization peptide. Each oligomerization region can comprise an RSV F HRA amino acid sequence. The complex can consist of the three RSV F ectodomain polypeptides and three oligomerization polypeptides. One or more of the oligomerization polypeptides can further comprise a functional region that is operably linked to the oligomerization region. The amino acid sequence of the RSV F ectodomain polypeptides provided in step (a) can be selected from the group consisting of: SEQ ID NO: 8 (Del21 Furx), SEQ ID NO: 3 (Furmt), SEQ ID NO: 4 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag, is omitted. Optionally, at least one of the RSV F ectodomain polypeptides can be a recombinant polypeptide that comprises a C-terminal 6-helix bundle forming moiety. Optionally, the C-terminal 6-helix bundle forming moiety can comprise a heptad repeat region of the fusion region of the fustion protein of an enveloped virus. The heptad repeat region can be the HRA or HRB from a Type I fusion protein of an enveloped virus. For example, the heptad repeat region can be RSV F HRA, RSV F HRB, or HIV gp41 HRA. The six-helix bundle can comprise the C-terminal 6 helix bundle forming moiety of three recombinant RSV F ectodomain polypeptides and the oligomerization region of each oligomerization peptide. The RSV F ectodomain polypeptides in the complex that is produced can be in the pre-fusion conformation. The RSV F ectodomain polypeptides in the complex that is produced can be characterized by a rounded shape when viewed in negatively stained electron micrographs. The RSV F ectodomain polypeptides in the complex that is produced can comprise prefusion epitopes that are not present on post-fusion forms of RSV F.

The invention also relates to a method for producing a respiratory syncytial virus F (RSV F) complex that comprises (a) providing RSV F protein ectodomain polypeptides that contain a C-terminal 6-helix bundle forming moiety, and (b) combining the RSV F ectodomain polypeptides under conditions suitable for the formation of a RSV F complex, whereby a RSV F complex is produced that comprises three RSV F ectodomain polypeptides and is characterized by a six-helix bundle formed by the C-terminal 6-helix bundle forming moiety and the endogenous HRB region.

The invention also relates to a respiratory syncytial virus (RSV F) complex produced by any of the methods described herein.

The invention also relates to an immunogenic composition that comprises a respiratory syncytial virus F (RSV F) complex as described herein.

The invention also relates to a method of inducing an immune response to RSV F in a subject that comprises administering an immunogenic composition to the subject.

### BRIEF DESCRIPTION OF THE DRAWINGS

FIG. 1A is a schematic of the wild type RSV F protein showing the signal sequence or signal peptide (SP), p27 linker region, fusion peptide (FP), HRA domain (HRA), HRB domain (HRB), transmembrane region (TM), and cytoplasmic tail (CT). The C-terminal bounds of the ectodomain can vary. FIG. 1B is a general schematic of the RSV F ectodomain construct in which the transmembrane domain and cytoplasmic tail have been removed and an optional HIS₆-tag (SEQ ID NO: 40) has been added to the C-terminus. It depicts the shared features with the schematics in FIG. 1A and the optional HIS₆-tag (HIS TAG) (SEQ ID NO: 40). Furin cleavage sites are present at amino acid positions 109/110 and 136/137. FIG. 1C shows the amino acid sequences of amino acids 100-150 of RSV F (wild type) (SEQ ID NO:25) and several proteins (Furmt-SEQ ID NO:3; Furdel-SEQ ID NO:4; Furx-SEQ ID NO:5; Furx R113Q, K123N, K124N-SEQ ID NO:6; Furx R113Q, K123Q, K124Q-SEQ ID NO:7; Delp21 furx-SEQ ID NO:8; Delp23 furx-SEQ ID NO:9; Delp23 furdel-SEQ ID NO:11; N-Term Furin-SEQ ID NO:12; C-term Furin-SEQ ID NO:13; Fusion Peptide Deletion 1-SEQ ID NO:26; and Factor Xa-SEQ ID NO:14) in which one or both furin cleavage sites and/or fusion peptide region were mutated or deleted. In FIG. 1C, the symbol “-” indicates that the amino acid at that position is deleted. For clarity, residue numbering in FIGS. 1A, 1B, and 1C is related to the wild type A2 strain RSV F beginning at the N-terminal signal peptide and is not altered in constructs containing amino acid deletions.

FIGS. 2A-2D show an alignment of the amino acid sequences of F proteins from several strains of RSV. The alignment was prepared using the algorithm disclosed by Corpet, Nucleic Acids Research, 1998, 16(22):10881-10890, using default parameters (Blossum 62 symbol comparison table, gap open penalty: 12, gap extension penalty: A2, F protein of the strain A2 (accession number AF035006) (SEQ ID NO: 27); CP52, F protein of the CP52 strain (accession number AF013255) (SEQ ID NO: 28); B, F protein of the B strain (accession number AF013254) (SEQ ID NO: 29); long, F protein of the long strain (accession number AY911262) strain (SEQ ID NO: 30), and 18537 strain, F protein of the 18537 strain (accession number Swiss Prot P13843) (SEQ ID NO: 31). A consensus of F protein sequences is also shown (SEQ ID NO: 24).

FIG. 3 is a schematic showing an in vitro trimerization process, whereby RSV F monomer solution containing HRB (the ectodomain peptides) are expressed and purified, then mixed with HRA peptides (the oligomerization peptides), inducing the formation of a six molecule complex that contains HRB from the F protein and HRA peptide in the form of an RSV monomer/trimer “head” and an artificial 6-helix bundle (A, B and C). Trimers are purified, and optionally trypsin can be used to cleave a cleavable monomer, which may allow the globular head of prefusion F to form (D and E).

### DETAILED DESCRIPTION OF THE INVENTION

The inventors discovered that producing recombinant RSV F polypeptides in the form of homotrimers, as they appear on the virion, requires cleavage of the RSV F polypeptides, and that RSV F polypeptide monomers are formed when the polypeptides are uncleaved. When the RSV F ectodomain is cleaved in vivo the protein forms trimers that bind to cellular debris, making purification difficult.

The inventors have developed an in vitro approach that uses oligomerizing peptides or inserted oligomerizing moieties to produce RSV F complexes in which all or a portion of the oligomerizing polypeptide or the inserted oligomerizing moieties forms a six-helix bundle with a portion of the RSV F polypeptide (e.g., HRB, HRA, and inserted sequence). Accordingly, in some aspects, the invention relates to soluble RSV F polypeptide complexes that contain three RSV F ectodomain polypeptides and three oligomerization polypeptides. As described herein, the complexes are stable and can conveniently be produced on a commercial scale. Stable complexes are able to produce immunogenic compositions in which the protein has a decreased tendency to aggregate or degrade, which provides a more predictable immune response when the composition is administered to a subject. In some embodiments, the structure of the RSV F ectodomain in the complex is in the pre-fusion conformation. The epitopes of the pre-fusion conformation may be better able to elicit antibodies that can recognize and neutralize natural virions. The invention also relates to methods for producing such complexes, immunogenic compositions comprising the complexes and methods of using the complexes and compositions.

### DEFINITIONS

The “post fusion conformation” of RSV F protein is a trimer characterized by the presence of a six-helix bundle, comprising 3 endogenous HRB and 3 endogenous HRA regions. Post-fusion conformations are further characterized by a cone-shape when viewed in negatively stained electron micrographs and/or by a lack of prefusion epitopes. See, e.g., Magro, M. et al., Proc. Natl. Acad. Sci. USA, 109(8):3089-94 (2012)).

The “pre-fusion conformation” of RSV F protein is a trimer in which the endogenous HRA regions do not interact with the endogenous HRB regions to form a six-helix bundle. A six-helix bundle may be present in the pre-fusion conformation, provided that the endogenous HRA regions are not a part of the six-helix bundle. Pre-fusion conformations are further characterized by a rounded shape when viewed in negatively stained electron micrographs, similar to that seen in the PIV5 pre-fusion F structure (See, e.g., Yin H S, et al. (2006) Nature 439(7072):38-44) and/or by prefusion epitopes that are not present on post-fusion conformations. See, e.g., Magro, M. et al., Proc. Natl. Acad. Sci. USA, 109(8):3089-94 (2012))

As used herein, the term “endogenous HRA region” refers to an HRA region that is present in a F polypeptide at substantially the same position as the HRA region in the amino acid sequence of the F0 form of the naturally occurring F protein. In the case of RSV F proteins, such as an RSV F ectodomain polypeptide or recombinant RSV F ectodomain polypeptide, the endogenous HRA region is from about amino acid 154 to about amino acid 206. Amino acid numbering is based on the sequence of wild type A2 strain of RSV F (SEQ ID NO: 1) including the signal peptide, and amino acid positions are assigned to residues that are deleted. For example, if the fusion peptide of RSV F is deleted in whole or in part, the deleted amino acids would be numbered so that the amino acids of HRA region have the same position numbers as in the wild type sequence.

As used herein, the term “inserted HRA region” refers to an HRA region that is present in a F polypeptide at a different position than the HRA region in the amino acid sequence of the F0 form of the naturally occurring F protein. For example, an RSV F polypeptide can contain an inserted HRA region, for example that is located carboxy terminally to the HRB region, and an endogenous HRA region.

As used herein, “RSV F ectodomain polypeptide” refers to an RSV F polypeptide that contains substantially the extracellular portion of mature RSV F protein, with or without the signal peptide (e.g., about amino acid 1 to about amino acid 524, or about amino acid 22 to about amino acid 524) but lacks the transmembrane domain and cytoplasmic tail of naturally occurring RSV F protein. The RSV F ectodomain polypeptide comprises an HRB domain.

As used herein, “cleaved RSV F ecto-domain polypeptide” refers to a RSV F ectodomain polypeptide that has been cleaved at one or more positions from about 101/102 to about 160/161 to produce two subunits, in which one of the subunits comprises F₁ and the other subunit comprises F₂.

As used herein, “C-terminal uncleaved RSV F ectodomain polypeptide” refers to an RSV F ectodomain polypeptide that is cleaved at one or more positions from about 101/102 to about 131/132, and is not cleaved at one or more positions from about 132/133 to about 160/161, to produce two subunits, in which one of the subunits comprises F₁ and the other subunit comprises F₂.

As used herein, “uncleaved RSV F ectodomain polypeptide” refers to an RSV F ectodomain polypeptide that is not cleaved at one or more positions from about 101/102 to about 160/161. An uncleaved RSV F ectodomain polypeptide can be, for example, a monomer or a trimer.

As used herein, a “purified” protein or polypeptide is a protein or polypeptide which is recombinantly or synthetically produced, or produced by its natural host, and has been isolated from other components of the recombinant or synthetic production system or natural host such that the amount of the protein relative to other macromolecular components present in a composition is substantially higher than that present in a crude preparation. In general, a purified protein will comprise at least about 50% of the protein in the preparation and more preferably at least about 75%, at least about 80%, at least about 85%, at least about 90%, at least about 95% of the protein in the preparation.

As used herein, “substantially free of lipids and lipoproteins” refers to compositions, proteins and polypeptides that are at least about 95% free of lipids and lipoproteins on a mass basis when protein and/or polypeptide (e.g., RSV F polypeptide) purity is observed on an SDS PAGE gel and total protein content is measured using either UV280 absorption or BCA analysis, and lipid and lipoprotein content is determined using the Phospholipase C assay (Wako, code no. 433-36201).

As used herein, “altered furin cleavage site” refers to the amino acid sequence at about positions 106-109 and at about positions 133-136 in naturally occurring RSV F protein that are recognized and cleaved by furin or furin-like proteases, but in an uncleaved RSV F protein ecto-domain polypeptide contains one or more amino acid replacements, one or more amino acid deletions, or a combination of one or more amino acid replacement and one or more amino acid deletion, so that an RSV F ecto-domain polypeptide that contains an altered furin cleavage site is secreted from a cell that produces it uncleaved at the altered furin cleavage site.

As used herein, “oligomerization polypeptide” refers to a polypeptide or polypeptide conjugate that is a separate molecule from the RSV F polypeptides described herein, and that contains an oligomerization region and optionally a functional region. The oligomerization region contains an amino acid sequence that can bind an RSV F ectodomain polypeptide and form a six-helix bundle with a corresponding portion of the RSV F ectodomain polypeptide. For example, when the oligomerization polypeptide comprises an RSV F HRA amino acid sequence, it can form a six-helix bundle with the endogenous HRB region of a RSV F polypeptide. When the oligomerization polypeptide contains an oligomerization region and a functional region, the two regions are operably linked so that the oligomerization region can form a six helix bundle with the RSV F ectodomain polypeptide and the functional region retains the desired functional activity.

As used herein, “C-terminal 6-helix bundle forming moiety” refers to a portion of a recombinant RSV F ectodomain polypeptide that can form a six-helix bundle and is 1) located C-terminally of the endogenous HRB region of naturally occurring RSV F protein, and 2) is not found in that location in naturally occurring RSV F protein. In one example, the C-terminal 6-helix bundle forming moiety is an HRA region of RSV F that is inserted C-terminally of the endogenous HRB region of RSV F, with or without the use of a linker sequence. A C-terminal 6-helix bundle forming moiety can form a six-helix bundle with one or more oligomerization polypeptides or with endogenous portions of a recombinant RSV F polypeptide.

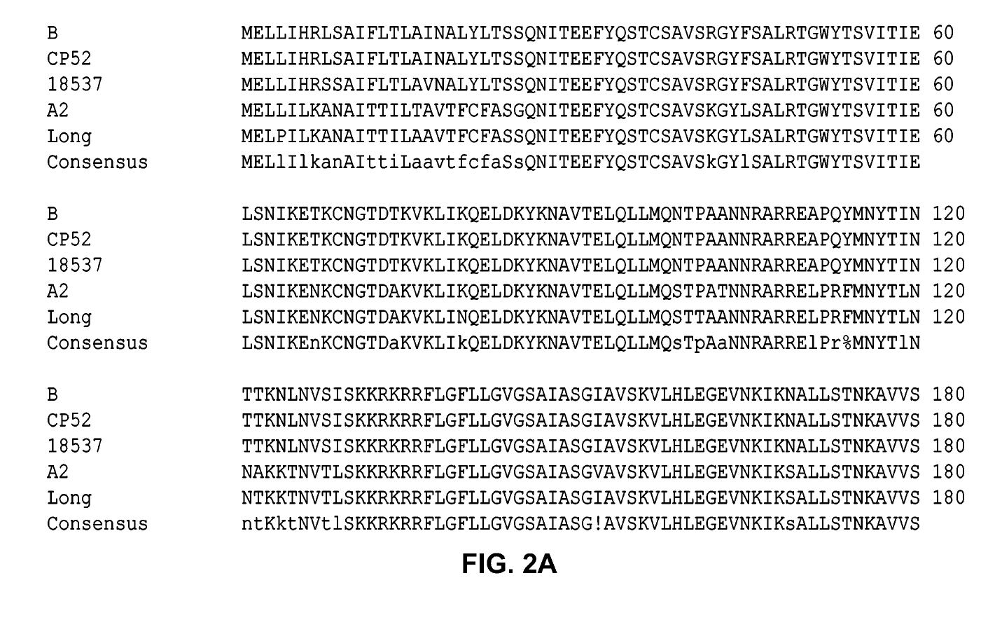

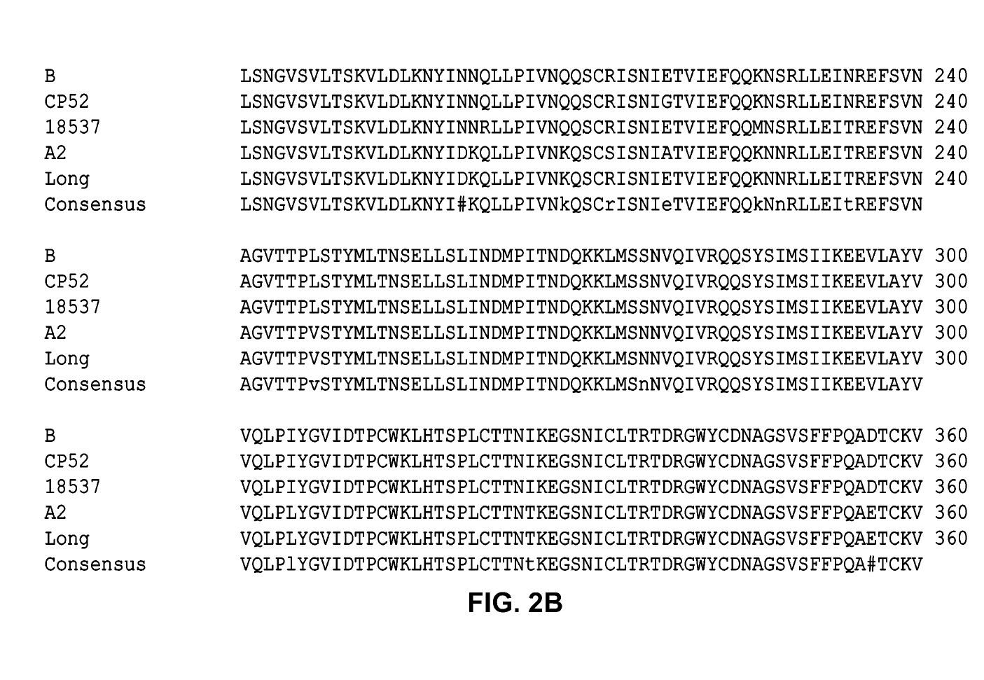

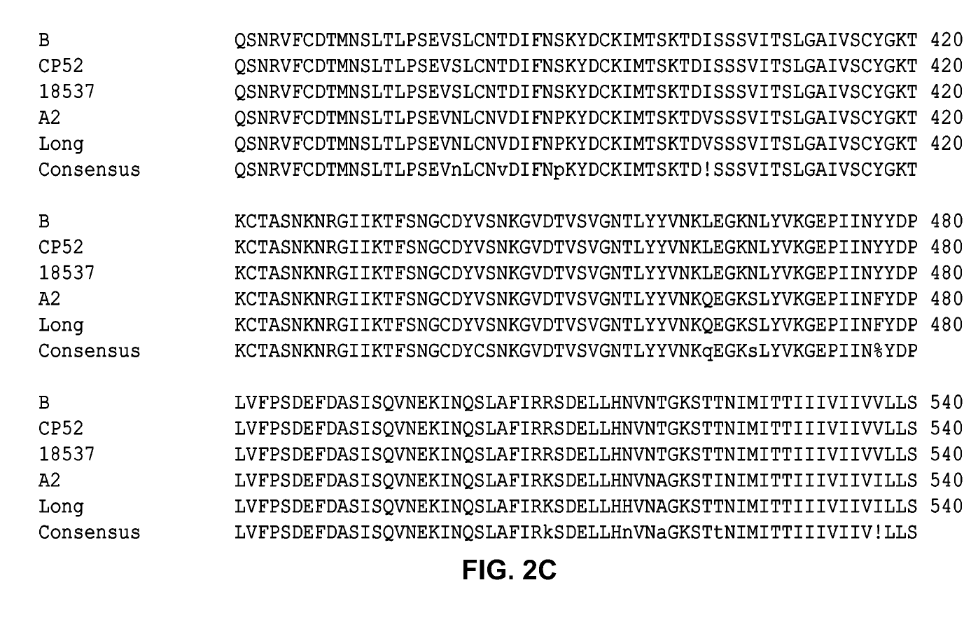

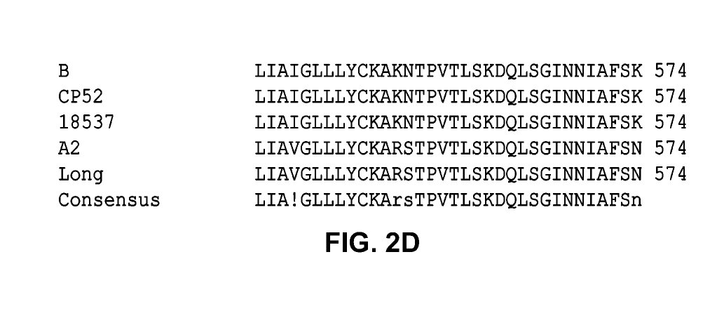

Features of RSV F protein ectodomains suitable for use in this invention are described herein with reference to particular amino acids that are identified by the position of the amino acid in the sequence of RSV F protein from the A2 strain (SEQ ID NO:1). RSV F protein ecto-domains can have the amino acid sequence of the F protein from the A2 strain or any other desired strain. When the F protein ectodomain from a strain other than the A2 strain is used, the amino acids of the F protein are to be numbered with reference to the numbering of the F protein from the A2 strain, with the insertion of gaps as needed. This can be achieved by aligning the sequence of any desired RSV F protein with the F protein of the strain A2, as shown herein for F proteins from the A2 strain, CP52 strain, B strain, long strain, and the 18537 strain. See, FIG. 2. Sequence alignments are preferably produced using the algorithm disclosed by Corpet, Nucleic Acids Research, 1998, 16(22):10881-10890, using default parameters (Blossum 62 symbol comparison table, gap open penalty: 12, gap extension penalty: 2).

The RSV F Glycoprotein

The F glycoprotein of RSV directs viral penetration by fusion between the virion envelope and the host cell plasma membrane. It is a type I single-pass integral membrane protein having four general domains: N-terminal ER-translocating signal sequence (SS), ectodomain (ED), transmembrane domain (TM), and a cytoplasmic tail (CT). CT contains a single palmitoylated cysteine residue. The sequence of F protein is highly conserved among RSV isolates, but is constantly evolving (Kim et al. (2007) J Med Virol 79: 820-828).

Unlike most paramyxoviruses, the F protein in RSV can mediate entry and syncytium formation independent of the other viral proteins (HN is usually necessary in addition to F in other paramyxoviruses).

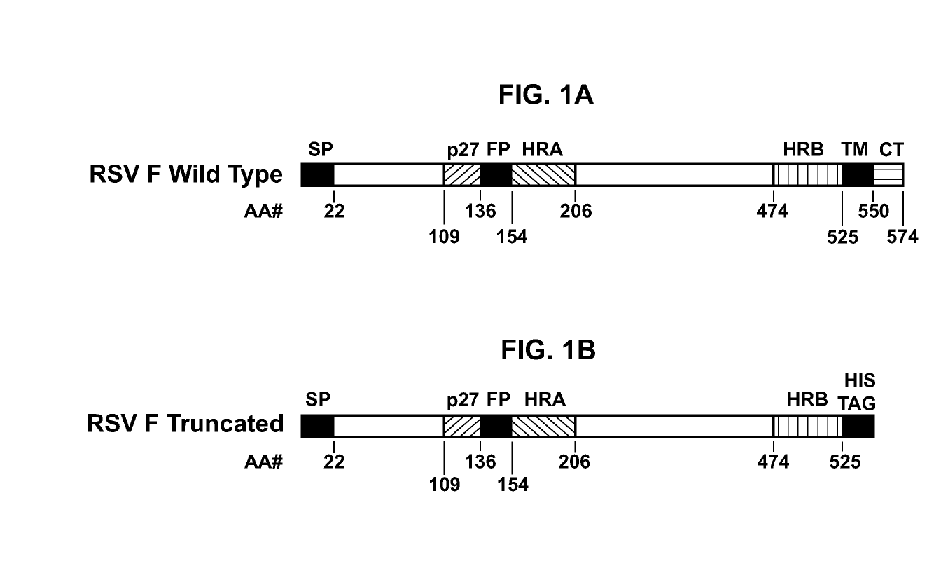

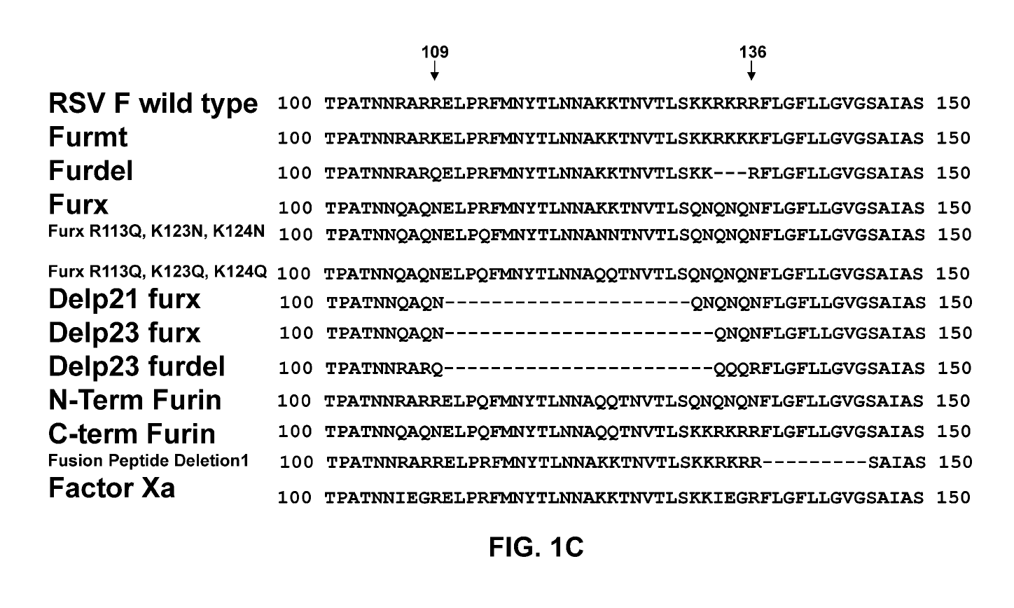

The hRSV F mRNA is translated into a 574 amino acid precursor protein designated F₀, which contains a signal peptide sequence at the N-terminus that is removed by a signal peptidase in the endoplasmic reticulum. F₀ is cleaved at two sites (a.a. 109/110 and 136/137) by cellular proteases (in particular furin) in the trans-Golgi, removing a short glycosylated intervening sequence and generating two subunits designated F₁ (˜50 kDa; C-terminus; residues 137-574) and F₂ (˜20 kDa; N-terminus; residues 1-109) (See, e.g., FIG. 1). F₁ contains a hydrophobic fusion peptide at its N-terminus and also two hydrophobic heptad-repeat regions (HRA and HRB). HRA is near the fusion peptide and HRB is near to the transmembrane domain (See, e.g., FIG. 1). The F₁-F₂ heterodimers are assembled as homotrimers in the virion.

RSV exists as a single serotype but has two antigenic subgroups: A and B. The F glycoproteins of the two groups are about 90% identical in amino acid sequence. The A subgroup, the B subgroup, or a combination or hybrid of both can be used in the invention. An example sequence for the A subgroup is SEQ ID NO: 1 (A2 strain; GenBank GI: 138251; Swiss Prot P03420), and for the B subgroup is SEQ ID NO: 2 (18537 strain; GI: 138250; Swiss Prot P13843). SEQ ID NO:1 and SEQ ID NO:2 are both 574 amino acid sequences. The signal peptide in A2 strain is a.a. 1-21, but in 18537 strain it is 1-22. In both sequences the TM domain is from about a.a. 530-550, but has alternatively been reported as 525-548.

| SEQ ID NO: 1 | | |
|---|---|---|
| 1 | MELLILKANAITTILTAVTFCFASGQNITEEFYQSTCSAVSKGYLSALRTGWYTSVITIE | 60 |
| 61 | LSNIKENKCNGTDAKVKLIKQELDKYKNAVTELQLLMQSTPPTNNRARRELPRFMNYTLN | 120 |
| 121 | NAKKTNVTLSKKRKRRFLGFLLGVGSAIASGVAVSKVLHLEGEVNKIKSALLSTNKAVVS | 180 |
| 181 | LSNGVSVLTSKVLDLKNYIDKQLLPIVNKQSCSISNIETVIEFQQKNNRLLEITREFSVN | 240 |
| 241 | AGVTTPVSTYMLTNSELLSLINDMPITNDQKKLMSNNVQIVRQQSYSIMSIIKEEVLAYV | 300 |
| 301 | VQLPLYGVIDTPCWKLHTSPLCTTNTKEGSNICLTRTDRGWYCDNAGSVSFFPQAETCKV | 360 |
| 361 | QSNRVFCDTMNSLTLPSEINLCNVDIFNPKYDCKIMTSKTDVSSSVITSLGAIVSCYGKT | 420 |
| 421 | KCTASNKNRGIIKTFSNGCDYVSNKGMDTVSVGNTLYYVNKQEGKSLYVKGEPIINFYDP | 480 |
| 481 | LVFPSDEFDASISQVNEKINQSLAFIRKSDELLHNVNAGKSTTNIMITTIIIVIIVILLS | 540 |
| 541 | LIAVGLLLYCKARSTPVTLSKDQLSGINNIAFSN | 574 |
| SEQ ID NO: 2 | | |
| 1 | MELLIHRSSAIFLTLAVNALYLTSSQNITEEFYQSTCSAVSRGYFSALRTGWYTSVITIE | 60 |
| 61 | LSNIKETKCNGTDTKVKLIKQELDKYKNAVTELQLLMQNTPAANNRARREAPQYMNYTIN | 120 |
| 121 | TTKNLNVSISKKRKRRFLGFLLGVGSAIASGIAVSKVLHLEGEVNKIKNALLSTNKAVVS | 180 |
| 181 | LSNGVSVLTSKVLDLKNYINNRLLPIVNQQSCRISNIETVIEFQQMNSRLLEITREFSVN | 240 |
| 241 | AGVTTPLSTYMLTNSELLSLINDMPITNDQKKLMSSNVQIVRQQSYSIMSIIKEEVLAYV | 300 |
| 301 | VQLPIYGVIDTPCWKLHTSPLCTTNIKEGSNICLTRTDRGWYCDNAGSVSFFPQADTCKV | 360 |
| 361 | QSNRVFCDTMNSLTLPSEVSLCNTDIFNSKYDCKIMTSKTDISSSVITSLGAIVSCYGKT | 420 |
| 421 | KCTASNKNRGIIKTFSNGCDYVSNKGVDTVSVGNTLYYVNKLEGKNLYVKGEPIINYYDP | 480 |
| 481 | LVFPSDEFDASISQVNEKINQSLAFIRRSDELLHNVNTGKSTTNIMITTIIIVIIVVLLS | 540 |
| 541 | LIAIGLLLYCKAKNTPVTLSKDQLSGINNIAFSK | 574 |

The invention may use any desired RSV F amino acid sequence, such as the amino acid sequence of SEQ ID NO: 1 or 2, or a sequence having identity to SEQ ID NO: 1 or 2. Typically it will have at least 75% identity to SEQ ID NO: 1 or 2 e.g., at least 80%, at least 85%, at least 90%, at least 95%, at least 97%, at least 98%, at least 99%, identity to SEQ ID NO:1 or 2. The sequence may be found naturally in RSV.

Preferably an ectodomain of F protein, in whole or in part, is used, which may comprise:

(i) a polypeptide comprising about amino acid 22-525 of SEQ ID NO: 1;

(ii) a polypeptide comprising about amino acids 23-525 of SEQ ID NO: 2;

(iii) a polypeptide comprising an amino acid sequence having at least 75% identity (e.g., at least 80%, at least 85%, at least 90%, at least 95%, at least 97%, at least 98%, at least 99% identity) to (i) or (ii); or

(iv) a polypeptide comprising a fragment of (i), (ii) or (iii), wherein the fragment comprises at least one F protein epitope. The fragment will usually be at least about 100 amino acids long, e.g., at least about 150, at least about 200, at least about 250, at least about 300, at least about 350, at least about 400, at least about 450 amino acids long.

The ectodomain can be an F₀ form with or without the signal peptide, or can comprises two separate peptide chains (e.g., an F₁ subunit and a F₂ subunit) that are associated with each other, for example, the subunits may be linked by a disulfide bridge. Accordingly, all or a portion of about amino acid 101 to about 161, such as amino acids 110-136, may be absent from the ectodomain. Thus the ectodomain, in whole or in part, can comprise:

(v) a first peptide chain and a second peptide chain that is associated with the first polypeptide chain, where the first peptide chain comprises an amino acid sequence having at least 75% identity (e.g., at least 80%, at least 85%, at least 90%, at least 95%, at least 97%, at least 98%, at least 99%, or even 100% identity) to about amino acid 22 to about amino acid 101 of SEQ ID NO: 1 or to about amino acid 23 to about amino acid 101 of SEQ ID NO: 2, and the second peptide chain comprises an amino acid sequence having at least 75% identity (e.g., at least 80%, at least 85%, at least 90%, at least 95%, at least 97%, at least 98%, at least 99%, or even 100% identity) to about amino acid 162 to about 525 of SEQ ID NO: 1 or to about amino acid 162 to 525 of SEQ ID NO: 2;

(vi) a first peptide chain and a second peptide chain that is associated with the first polypeptide chain, where the first peptide chain comprises an amino acid sequence comprising a fragment of about amino acid 22 to about amino acid 101 of SEQ ID NO: 1 or of about amino acid 23 to about amino acid 109 of SEQ ID NO: 2, and the second peptide chain comprises a fragment of about amino acid 162 to about amino acid 525 of SEQ ID NO: 1 or of about amino acid 161 to about amino acid 525 of SEQ ID NO: 2. One or both of the fragments will comprise at least one F protein epitope. The fragment in the first peptide chain will usually be at least 20 amino acids long, e.g., at least 30, at least 40, at least 50, at least 60, at least 70, at least 80 amino acids long. The fragment in the second peptide chain will usually be at least 100 amino acids long, e.g., at least 150, at least 200, at least 250, at least 300, at least 350, at least 400, at least 450 amino acids long; or

(vii) a molecule obtainable by furin digestion of (i), (ii), (iii) or (iv).

Thus an amino acid sequence used with the invention may be found naturally within RSV F protein (e.g., a soluble RSV F protein lacking TM and CT, about amino acids 522-574 of SEQ ID NOS: 1 or 2), and/or it may have one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) single amino acid mutations (insertions, deletions or substitutions) relative to a natural RSV sequence. For instance, it is known to mutate F proteins to eliminate their furin cleavage sequences, thereby preventing intracellular processing. In certain embodiments, the RSV F protein lacks TM and CT (about amino acids 522-574 of SEQ ID NOS: 1 or 2) and contains one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) single amino acid mutations (insertions, deletions or substitutions) relative to a natural RSV sequence.

RSV F polypeptides or proteins may contain one or more mutations that prevent cleavage at one or both of the furin cleavage sites (i.e., amino acids 109 and 136 of SEQ ID NOS: 1 and 2). RSV F ectodomain polypeptides that contain such mutations are not cleaved in vivo by cells that produce the polypeptides and are produced as monomers. Examples of suitable furin cleavage mutations include replacement of amino acid residues 106-109 of SEQ ID NO: 1 or 2 with RARK (SEQ ID NO: 32), RARQ (SEQ ID NO: 33), QAQN (SEQ ID NO: 34), or IEGR (SEQ ID NO: 35). Alternatively, or in addition, amino acid residues 133-136 of SEQ ID NO: 1 or 2 can be replaced with RKKK (SEQ ID NO: 36), ΔΔΔR, QNQN (SEQ ID NO: 37), QQQR (SEQ ID NO: 38) or IEGR (SEQ ID NO: 39). (Δ indicates that the amino acid residue has been deleted.) These mutations can be combined, if desired, with other mutations described herein or known in the art, such as mutations in the p27 region (amino acids 110-136 of SEQ ID NOS: 1 or 2), including deletion of the p27 region in whole or in part.

Generally, the amino acid sequence of an uncleaved RSV F protein ecto-domain is altered to prevent cleavage at the furin cleavage sites at about position 109/110 and about position 136/137, but contains a naturally occurring or inserted protease cleavage site, that when cleaved produce a F₁ subunit and a F₂ subunit. For example, the uncleaved RSV F protein ectodomain polypeptide can have an amino acid sequence that is altered to prevent cleavage at the furin cleavage sites at about position 109/110 and about position 136/137, but contain one or more naturally occurring or inserted protease cleavage sites from about position 101 to about position 161.

A variety of particular amino acid sequences that will allow uncleaved RSV F protein ecto-domain polypeptides to be produced and expressed by host cells, including amino acid sequences that are not cleaved at the furin cleavage sites at about position 109/110 and about position 136/137 can be readily designed and envisioned by a person of ordinary skill in the art. In general, one or more amino acids that are part of or are located nearby the furin cleavage sites at about position 109/110 and about position 136/137 are independently replaced or deleted. Some amino acid substitutions and deletions that are suitable to prevent cleavage of RSV F protein ecto-domain polypeptides are known. For example, the substitutions R108N, R109N, R108N/R109N, which inhibit cleavage at 109/110, and the substitution K131Q or the deletion of the amino acids at positions 131-134, which inhibit cleavage at 136/137, have been described Gonzalez-Reyes et al., Proc. Natl. Acad. Sci. USA, 98:9859-9864 (2001). An uncleaved RSV F ectodomain polypeptide that contains the amino acid substitutions R108N/R109N/K131Q/R133Q/R135Q/R136Q has been described. Ruiz-Arguello et al., J. Gen. Virol. 85:3677687 (2004). As described herein, additional RSV F protein amino acid sequences that result in the RSV F ecto-domain polypeptide being secreted from a host cell uncleaved contain altered furin cleavage sites, e.g., alter amino acid sequences at about positions 106-109 and at about positions 133-136. The altered furin cleavage sites contain at least one amino acid substitution or deletion at about positions 106-109, and at least one amino acid substitution or deletion at about positions 133-136.

Similarly, a variety of particular amino acid sequences of uncleaved RSV F protein ectodomain polypeptides that contain a protease cleavage site (e.g., naturally occurring or inserted) that when cleaved produce a first subunit that comprises an F₁ and a second subunit that comprises F₂ can be readily designed and envisioned. For example, the amino acid sequence of RSV F protein from about position 101 to about position 161 contains trypsin cleavage sites, and one or more of the trypsin cleavage sites can be cleaved, e.g., in vitro, by trypsin to generate F₁ and F₂ subunits. If desired, one or more suitable protease recognition sites can be inserted into the uncleaved RSV F protein ecto-domain polypeptide, for example, between about positions 101 to about position 161. The inserted protease recognition sites can be cleaved using the appropriate protease to generate F₁ and F₂ subunits.

In particular embodiments, the sequence of amino acid residue 100-150 of the RSV F polypeptide or protein, such as SEQ ID NO:1, SEQ ID NO:2, or the soluble ecto domains thereof, is

(Furmt)
(SEQ ID NO: 3)
TPATNNRARKELPRFMNYTLNNAKKTNVTLSKKRKKKFLGFLLGVGSAIA
S
(Furdel)
(SEQ ID NO: 4)
TPATNNRARQELPRFMNYTLNNAKKTNVTLSKK---RFLGFLLGVGSAIA
S
(Furx)
(SEQ ID NO: 5)
TPATNNQAQNELPRFMNYTLNNAKKTNVTLSQNQNQNFLGFLLGVGSAIA
S
(Furx R113Q, K123N, K124N)
(SEQ ID NO: 6)
TPATNNQAQNELPQFMNYTLNNANNTNVTLSQNQNQNFLGFLLGVGSAIA
S
(Furx R113Q, K123Q, K124Q))
(SEQ ID NO: 7)
TPATNNQAQNELPQFMNYTLNNAQQTNVTLSQNQNQNFLGFLLGVGSAIA
S
(Delp2lFurx)
(SEQ ID NO: 8)
TPATNNQAQN---------------------QNQNQNFLGFLLGVGSAIA
S
(Delp23Furx)
(SEQ ID NO: 9)
TPATNNQAQN-----------------------QNQNFLGFLLGVGSAIA
S
(Delp21 furdel)
(SEQ ID NO: 10)
TPATNNRARQ---------------------QNQQQRFLGFLLGVGSAIA
S
(Delp23furdel)
(SEQ ID NO: 11)
TPATNNRARQ-----------------------QQQRFLGFLLGVGSAIA
S
(Nterm Furin)
(SEQ ID NO: 12)
TPATNNRARRELPQFMNYTLNNAQQTNVTLSQNQNQNFLGFLLGVGSAIA
S
(Cterm Furin)
(SEQ ID NO: 13)
TPATNNQAQNELPQFMNYTLNNAQQTNVTLSKKRKRRFLGFLLGVGSAIA
S
(Factor Xa)
(SEQ ID NO: 14)
TPATNNIEGRELPRFMNYTLNNAKKTNVTLSKKIEGRFLGFLLGVGSAIA
S;
or
(WO 2010/077717)
(SEQ ID NO: 15)
TPPTNNRARRELPRFMNYTLNNAKKTNVTLSKKRKRR----------AIA
S
wherein the symbol “-” indicates that the amino
acid at that position is deleted.

### RSV F Complexes

The complexes contain an RSV F ectodomain trimer and are characterized by a six-helix bundle, with the proviso that the endogenous HRA is not part of the six-helix bundle.

In one aspect, the complexes may contain an RSV F ectodomain trimer in the form of a complex that contains three RSV F ectodomain polypeptides and at least one oligomerization polypeptide. The oligomerization polypeptide contains an oligomerization region or moiety that can bind with portions of RSV F ectodomain polypeptides to form a six-helix bundle. Thus, the complex contains a six-helix bundle that is formed by a portion of the RSV F ectodomain polypeptides and all or a portion of the oligomerization polypeptides.

The RSV F ectodomain contains portions that are capable of forming a six-helix bundle. For example, the HRB region of an RSV F ectodomain polypeptide can form a six-helix bundle with an oligomerization polypeptide that contains the amino acid sequence of the HRA region of RSV F.

If desired, one or more of the RSV F ectodomains present in the complexes described herein can be a recombinant RSV F ectodomain polypeptide that includes an inserted C-terminal 6-helix bundle forming moiety. Such recombinant RSV F ectodomain polypeptides can be prepared using methods that are conventional in the art. The C-terminal 6-helix bundle forming moiety can be from RSV F, but is present at a C-terminal location that is different (or in addition to) the location in which the moiety appears in naturally occurring RSV F. In one example, the C-terminal 6-helix bundle forming moiety is the HRA region of RSV F. Such a recombinant RSV F ectodomain polypeptide can form a six-helix bundle with an oligomerization polypeptide that contains the amino acid sequence of the HRB region of RSV F. Alternatively, the C-terminal 6-helix bundle forming moiety can be an exogenous moiety that is obtained from a protein other than RSV F, such as the HRA region of HIV gp41. Many six-helix bundle forming polypeptides are well-known in the art, such as the heptad repeat regions (e.g., HRA and HRB) of Type I fusion proteins of enveloped viruses, such as RSV F, PIV and the like. See, e.g., Weissenhorm et al., FEBS Letters 581: 2150-2155 (2007), Table 1.

The oligomerization polypeptide comprises an oligomerization region that can bind with a portion of the ectodomain of an RSV F polypeptide, e.g., HRB or an inserted C-terminal 6-helix bundle forming moiety, and thereby cause the complex to form. Many suitable polypeptide sequences that are suitable for use as oligomerization regions are well known in the art, such as the heptad repeat regions (e.g., HRA and HRB) of the fusion proteins of enveloped viruses such as RSV F, PIV and the like.

For example, when the RSV F ectodomain polypeptide comprises HRB, the oligomerization region can contain the amino acid sequence of RSV F HRA. Similarly, when the recombinant RSV F ectodomain polypeptide comprises a C-terminal 6-helix bundle forming moiety that is the HRA region of RSV F or HRA region of HIV gp41, for example, the oligomerization region can be the HRB region of RSV F or the HRB region of HIV gp41, respectively.

If desired, the oligomerization polypeptide can further comprise a functional region that is operably linked to the oligomerization region. Suitable methods for producing operable linkages between a polypeptide (i.e., the oligomerization region) and a desired functional region, such as another polypeptide, a lipid, a synthetic polymer, are well known in the art. For example, the oligomerization polypeptide can be a polypeptide in which an amino acid sequence comprising the oligomerization region and an amino acid sequence comprising the functional region are components of a contiguous polypeptide chain, with or without an intervening linker sequence. In one embodiment, the oligomerization polypeptide can be expressed and purified as a fusion of the oligomerization peptide and the additional functional region. For example, the oligomerization polypeptide may comprise the RSV F HRA region and be fused to the RSV G central domain, with or without an intervening linker sequence. Additionally, two polypeptides or a polypeptide and another molecule (e.g., a lipid, a synthetic polymer) can be chemically conjugated directly or through a linker using a variety of known approaches. See, e.g., Hermanson, G. T., Bioconjugate Techniques, 2nd Edition, Academic Press, Inc. 2008.

Suitable functional regions include all or a portion of an immunogenic carrier protein, an antigen, a particle forming polypeptide (e.g., viral particle or a non-infectious virus-like particle), a lipid, and polypeptides that can associate the oligomerization polypeptide with a liposome or particle (e.g., hydrophobic peptides, such as a transmembrane region, or a polypeptide that forms a coiled coil). When the functional region contains a portion of an immunogenic carrier protein, an antigen, a particle forming peptide, a lipid, or a polypeptide that can associate the oligomerization polypeptide with a liposome or particle, the portion that is contained is sufficient for the desired function. For example, when the oligomerization polypeptide contains a portion of an immunogenic carrier protein, the portion is sufficient to improve the immunogenicity of the RSV F complex. Similarly, when the oligomerization polypeptide contains a portion of an antigen, the portion is sufficient to induce an immune response.

Suitable immunogenic carrier proteins are well-known in the art and include, for example, albumin, keyhole limpet hemocyanin, tetanus toxoid, diphtheria toxoid, CRM197, rEPA (nontoxic Pseudomonas aeruginosa ExoProteinA), non-typeable Haemophilus influenzae protein D (NTHiD), N19 polyepitope and the like.

Suitable antigens are well-known in the art and include any antigen from a pathogen (e.g., a viral, bacterial or fungal pathogen). Exemplary antigens include, for example, RSV proteins such as RSV F and RSV G, HIV proteins such as HIV gp41, influenza proteins such as hemagglutinin, and paramyxovirus proteins such as the fusion protein of hPIV5, hPIV3 or Newcastle Disease virus.

Suitable particle forming peptides are well-known in the art and include, for example, viral polypeptides that form viral particles, such as capsid proteins from rotavirus (VP4 and VP7), nodavirus, norovirus, human papillomavirus (L1 and L2)), parvovirus B19 (VP1 and VP2), hepatitis B virus (core protein), as well as monomers of self-assembling peptide nanoparticles, e.g., as described in Untied States Patent Application Publication No. 2011/0020378. In one embodiment, the oligomerizing polypeptide comprises an oligomerization region that is operably linked to a monomer of a self-assembling peptide nanoparticle as described in United States Patent Application Publication No. 2011/0020378.

Suitable lipids are well-known in the art and include, for example, fatty acids, sterols, mono-, di- and triglycerides and phospholipids. Such lipids can anchor RSV F complexes that contain them to liposomes, membranes, oil in water emulsion droplets and other structures. Exemplary lipids that can be used as a functional region of an oligomerization polypeptide include myristoyl, palmitoyl, glycophosphatidylinositol, pegylated lipids, neutral lipid, and nanodisks. Advantageously, myristoyl, palmitoyl, and glycophosphatidylinositol can be incorporated into the oligomerization polypeptide in vivo by expression of a construct that enclodes the oligomerization polypeptide in a suitable host cell.

A variety of suitable polypeptides that can associate the oligomerization polypeptide with a liposome or particle can be included in the oligomerization polypeptide and are well-known in the art (see, e.g., WO2010/009277 and WO2010/009065). For example, hydrophobic polypeptides e.g., a transmembrane region or a fusion peptide, that associate with or insert into liposomes or lipid nanoparticles can be used. Polypeptides that form a coiled coil can be used to link the oligomerization polypeptide to other structures that contain a coiled coil-forming peptide, e.g., a synthetic nanoparticle or liposome; or viral polypeptides, viral particles. In one embodiment, the oligomerizing polypeptide comprises an oligomerization region that is operably linked to coiled coil forming peptide that can bind the complex to a self-assembling peptide nanoparticle, as described in United States Patent Application Publication No. 2011/0020378.

In some embodiments, the invention is a RSV F complex that contains three RSV F ectodomain polypeptides and three oligomerization polypeptides. The complex is characterized by a six-helix bundle formed by the HRB region of each of the three RSV F ectodomain polypeptides and all or a portion (i.e., the oligomerization region) of each of the three oligomerization polypeptides. In this type of complex, the oligomerization region of each oligomerization peptide preferably comprises the amino acid sequence of the HRA region of RSV F.

In particular embodiments, the RSV F ectodomain polypeptides are recombinant and each comprises a C-terminal 6-helix bundle forming moiety. The complex in these embodiments is characterized by a six-helix bundle formed by the C-terminal 6-helix bundle forming moiety of each of the three RSV F ectodomain polypeptides and all or a portion (i.e., the oligomerization region) of each of the three oligomerization polypeptides.

In other aspects, the complex does not include an oligomerization polypeptide. The complexes of this aspect contain three RSV F ectodomain polypeptides, at least one of which contains a C-terminal 6-helix bundle forming moiety. The complex is characterized by a six-helix bundle that is formed by the C-terminal 6-helix bundle forming moiety and endogenous portions of the RSV F ectodomain polypeptides. For example, such a complex can contain one, two or three recombinant RSV F ectodomain polypeptides that contain a C-terminal 6-helix bundle forming moiety, such as an inserted RSV F HRA amino acid sequence. The C-terminal 6-helix bundle forming moiety (e.g., inserted HRA sequence) can form a six-helix bundle with the endogenous (e.g., HRB) region. Without wishing to be bound by any particular theory, it is believed that the C-terminal 6-helix bundle forming moiety can fold back on the RSV F polypeptide to interact with endogenous portions of the polypeptide and form the six-helix bundle. Accordingly, in this aspect linker sequences can be included to permit the C-terminal 6-helix bundle to interact with endogenous portions of the polypeptide and form the six-helix bundle.

One or more of the RSV F ectodomain polypeptides in the complex can be an uncleaved RSV F ectodomain polypeptide, and the remaining can be a cleaved RSV F ectodomain polypeptide. In certain preferred embodiments, each of the RSV F ectodomain polypeptides in the complex contains one or more altered furin cleavage sites.

In more particular embodiments, the amino acid sequence of the RSV F ectodomain polypeptides is selected from the group consisting of: SEQ ID NO: 8 (Del21 Furx), SEQ ID NO: 3 (Furmt), SEQ ID NO: 4 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag, is omitted.

In particular embodiments, the amino acid sequence of the oligomerization polypeptide is selected from the group consisting of: SEQ ID NO:16 (RSV HRA, an oligomerization peptide of HRA), SEQ ID NO:17 (HRA_short, an oligomerization peptide that is slightly shorter than RSV HRA, SEQ ID NO:16), or any of the forgoing in which the GST sequence, cleavage sequence and/or linker sequence is omitted. In SEQ ID NOS:16-17, the sequence in normal text is glutathione S-transferasse (GST), the underlined sequence is a cleavage sequence, the double underlined sequence is a linker, and the bold sequence is HRA.

>RSV HRA
(SEQ ID NO: 16)
MHHHHHHGSMSPILGYWKIKGLVQPTRLLLEYLEEKYEEHLYERDEGDKWRNKKFELGLEFP
NLPYYIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCLDAFPKLV
CFKKRIEAIPQIDKYLKSSKYIAWPLQGWQATFGGGDHPPKSDLVPRGSGSLEVLFQGPGGS
AGSGLEGEVNKIKSALLSTNKAVVSLSNGVSVLTSKVLDLKNYIDKQLLPIV
>HRA_short
(SEQ ID NO: 17)
MHHHHHHGSMSPILGYWKIKGLVQPTRLLLEYLEEKYEEHLYERDEGDKWRNKKFELGLEFP
NLPYYIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCLDAFPKLV
CFKKRIEAIPQIDKYLKSSKYIAWPLQGWQATFGGGDHPPKSDLVPRGSGSLEVLFQGPGGS
AGSGLEGEVNKIKSALLSTNKAVVSLSNGVSVLTSKVLDLKN

In particular embodiments, the RSV F complex contains an RSV F ectodomain polypeptide and an oligomerization polypeptide that includes a functional region, such as an antigen. For example, the oligomerization polypeptide can comprise the amino acid sequence SEQ ID NO:18 (RSV Gb CC HRA short, in which an HRA oligomerization sequence is fused to the central domain of RSV G from strain b), SEQ ID NO:19 (RSV Ga CC HRA short, in which an HRA oligomerization sequence is fused to the central domain of RSV G from strain a), SEQ ID NO:20 (RSV Gb CC HRB, in which an HRB oligomerization sequence is fused to the central domain of RSV G from strain b), SEQ ID NO:21 (RSV Ga CC HRB, in which an HRB oligomerization sequence is fused to the central domain of RSV G from strain a), or any of the foregoing in which the glutathione S-transferase (GST) sequence, cleavage sequence and/or amino terminal linker sequence is omitted. In SEQ ID NOS: 18-21, the sequence in normal text is GST, the underlined sequence is a cleavage sequence, the double underlined sequences are linkers, the sequence that is dotted underlined is the Gb or Ga sequence, and the bold sequence is HRA or HRB.

>RSV Gb CC HRA short
(SEQ ID NO: 18)
MHHHHHHGSMSPILGYWKIKGLVQPTRLLLEYLEEKYEEHLYERDEGDKWRNKKFELGLEFP
NLPYYIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCLDAFPKLV
CFKKRIEAIPQIDKYLKSSKYIAWPLQGWQATFGGGDHPPKSDLVPRGSGSLEVLFQGPGGS

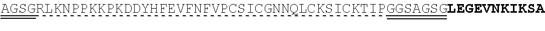

LLSTNKAVVSLSNGVSVLTSKVLDLKN
>RSV Ga CC HRA short
(SEQ ID NO: 19)
MHHHHHHGSMSPILGYWKIKGLVQPTRLLLEYLEEKYEEHLYERDEGDKWRNKKFELGLEFP
NLPYYIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCLDAFPKLV
CFKKRIEAIPQIDKYLKSSKYIAWPLQGWQATFGGGDHPPKSDLVPRGSGSLEVLFQGPGGS

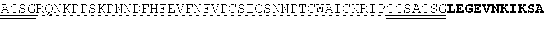

LLSTNKAVVSLSNGVSVLTSKVLDLKN
>RSV Gb CC HRB
(SEQ ID NO: 20)
MHHHHHHGSMSPILGYWKIKGLVQPTRLLLEYLEEKYEEHLYERDEGDKWRNKKFELGLEF
PNLPYYIDGDVKLTQSMAIIRYIADKHNMLGGCPKERAEISMLEGAVLDIRYGVSRIAYSK
DFETLKVDFLSKLPEMLKMFEDRLCHKTYLNGDHVTHPDFMLYDALDVVLYMDPMCLDAFP
KLVCFKKRIEAIPQIDKYLKSSKYIAWPLQGWQATFGGGDHPPKSDLVPRGSGSLEVLFQG

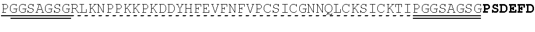

ASISQVNEKINQSLAFIRKSDELLHNVN
>RSV Ga CC HRB
(SEQ ID NO: 21)
MHHHHHHGSMSPILGYWKIKGLVQPTRLLLEYLEEKYEEHLYERDEGDKWRNKKFELGLEF
PNLPYYIDGDVKLTQSMAIIRYIADKHNMLGGCPKERAEISMLEGAVLDIRYGVSRIAYSK
DFETLKVDFLSKLPEMLKMFEDRLCHKTYLNGDHVTHPDFMLYDALDVVLYMDPMCLDAFP
KLVCFKKRIEAIPQIDKYLKSSKYIAWPLQGWQATFGGGDHPPKSDLVPRGSGSLEVLFQG

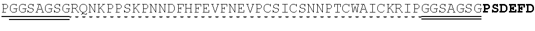

ASISQVNEKINQSLAFIRKSDELLHNVN

In other particular embodiments, the RSV F complex comprises an RSV F ectodomain construct selected from the group consisting of SEQ ID NO:22 (RSV F delP23 furdel Truncated HRA HIS), SEQ ID NO:23 (RSV F delP23 furdel C509C510 C481C489 HRA HIS) or any one of the foregoing in which the HIS tag and/or linker are omitted. In SEQ ID NOS:22-23 the sequence in normal text is an RSV F ectodomain sequence, the underlined sequence is an inserted C-terminal HRA sequence, the sequence that is double underlined is a linker, and the bold sequence is the HIS tag. SEQ ID NO:23 also includes introduced cysteines at positions 481, 489, 509 and 510.

>RSV F delP23 furdel Truncated HRA HIS
(SEQ ID NO: 22)
MELLILKANAITTILTAVTFCFASGQNITEEFYQSTCSAVSKGYLSALRTGWYTSVITIEL
SNIKENKCNGTDAKVKLIKQELDKYKNAVTELQLLMQSTPATNNRARQ-------------
----------
QQQRFLGFLLGVGSAIASGVAVSKVLHLEGEVNKIKSALLSTNKAVVSLSNGVSVLTSKVL
DLKNYIDKQLLPIVNKQSCSISNIETVIEFQQKNNRLLEITREFSVNAGVTTPVSTYMLTN
SELLSLINDMPITNDQKKLMSNNVQIVRQQSYSIMSIIKEEVLAYVVQLPLYGVIDTPCWK
LHTSPLCTTNTKEGSNICLTRTDRGWYCDNAGSVSFFPQAETCKVQSNRVFCDTMNSLTLP
SEVNLCNVDIFNPKYDCKIMTSKTDVSSSVITSLGAIVSCYGKTKCTASNKNRGIIKTFSN
GCDYVSNKGVDTVSVGNTLYYVNKQEGKSLYVKGEPIINFYDPLVFPSDEFDASISQVNEK
INQSLAFIRKSDELLHNLEGEVNKIKSALLSTNKAVVSLSNGVSVLTSKVLDLKNGGSAGS
GHHHHHH
>RSV F delP23 furdel C509C510 C481C489 HRA HIS
(SEQ ID NO: 23)
MELLILKANAITTILTAVTFCFASGQNITEEFYQSTCSAVSKGYLSALRTGWYTSVITIEL
SNIKENKCNGTDAKVKLIKQELDKYKNAVTELQLLMQSTPATNNRARQ-------------
----------
QQQRFLGFLLGVGSAIASGVAVSKVLHLEGEVNKIKSALLSTNKAVVSLSNGVSVLTSKVL
DLKNYIDKQLLPIVNKQSCSISNIETVIEFQQKNNRLLEITREFSVNAGVTTPVSTYMLTN
SELLSLINDMPITNDQKKLMSNNVQIVRQQSYSIMSIIKEEVLAYVVQLPLYGVIDTPCWK
LHTSPLCTTNTKEGSNICLTRTDRGWYCDNAGSVSFFPQAETCKVQSNRVFCDTMNSLTLP
SEVNLCNVDIFNPKYDCKIMTSKTDVSSSVITSLGAIVSCYGKTKCTASNKNRGIIKTFSN
GCDYVSNKGVDTVSVGNTLYYVNKQEGKSLYVKGEPIINFYDPLCFPSDEFCASISQVNEK
INQSLAFIRKCCELLHNLEGEVNKIKSALLSTNKAVVSLSNGVSVLTSKVLDLKNGGSAGS
GHHHHHH

In preferred embodiments, the RSV F ectodomain polypeptides in the complex are in the pre-fusion conformation. Without wishing to be bound by any particular theory, it is believed that the prefusion form of the RSV F trimer is stabilized in the complexes described herein because the oligomerization polypeptide induces complex formation and prevents the HRB and HRA regions of the RSV F protein from interacting. The interaction of the HRB and native HRA region of the RSV F protein leads to refolding into the post fusion form.

In other preferred embodiments, the complex is characterized by a rounded shape when viewed in negatively stained electron micrographs.

In other preferred embodiments, the complex comprises prefusion epitopes that are not present on post-fusion forms of RSV F.

Optionally, additional cysteine residues may be inserted into the HRB region to form disulfide bonds and further stabilize the RSV F complexes described herein.

### Methods for Preparing Complexes

The invention also relates to methods for producing the RSV F complexes described herein. In one aspect, the invention relates to methods for producing a RSV F complex that comprises three RSV F ectodomain polypeptides, three oligomerization polypeptides, and is characterized by a six-helix bundle. The method includes a) providing RSV F ectodomain polypeptides and oligomerization polypeptides, and b) combining the RSV F ectodomain polypeptides and oligomerization polypeptides under conditions suitable for the formation of an RSV F complex, whereby a RSV F complex is produced that comprises three RSV F ectodomain polypeptides, three oligomerization polypeptides, and is characterized by a six-helix bundle. As described herein, the six-helix bundle is formed by a portion of the RSV F ectodomain polypeptides and all or a portion of the oligomerization polypeptides.

If desired, one or more of the RSV F ectodomain polypeptides can be a recombinant RSV F ectodomain polypeptide that includes an inserted C-terminal 6-helix bundle forming moiety, such as the HRA region of RSV F or the HRA region of HIV gp41, for example. In this practice of the method, the oligomerization polypeptide comprises an oligomerization region that can bind with a portion of the RSV F ectodomain polypeptide, e.g., HRB or an inserted C-terminal 6-helix bundle forming moiety, and thereby cause the complex to form.

Optionally, the method can further comprise the step c) cleaving the RSV F protein ectodomain polypeptides in the produced complex with a suitable protease, whereby a RSV F complex is produced that comprises three cleaved RSV F ectodomain polypeptides, three of said oligomerization polypeptides, and is characterized by a six-helix bundle.

The complex that is formed using the method contains three RSV F ectodomain polypeptides and three oligomerization polypeptides. Thus, stoichiometric amounts of these polypeptides can be used in the method. However, excess oligomerization polypeptides can be used, and in practice 10-fold molar excess or more of oligomerization polypeptides can be used. The RSV F ectodomain polypeptides and three oligomerization polypeptides are combined under suitable conditions for the formation of the RSV F complex. Generally the RSV F ectodomain polypeptides and oligomerization polypeptides are combined in a buffered aqueous solution (e.g., pH about 5 to about 9). If desired, mild denaturing conditions can be used, such as, by including urea, small amounts of organic solvents or heat to mildly denature the RSV F ectodomain polypeptides.

Again, without wishing to be bound by any particular theory, it is believed that the method described herein is suitable for producing stable complexes in which the RSV F ectodomain polypeptides are in the pre-fusion conformation.

Any suitable preparation of RSV F ectodomain polypeptides and oligomerization polypeptides can be used in the method. For example, conditioned cell culture media that contains the desired polypeptide can be used in the method. However, it is preferable to use purified RSV F ectodomain polypeptides and oligomerization polypeptides in the method.

The use of uncleaved RSV F ectodomain polypeptides in the method provides advantages. As described herein, it has been discovered that cleavage of RSV F polypeptides in vivo of native RSV F ectodomains results in production of post-fusion ectodomains that are hydrophobic, aggregated, and difficult to purify. Cleavage in vivo of RSV F polypeptides with engineered features designed to stabilize the pre-fusion form results in poor yields or unprocessed/misfolded RSV F proteins. However, RSV F ectodomain polypeptides that are not cleaved in vivo are produced in good yield as monomers and when the fusion peptide is altered in these ectodomain polypeptides the protein can be soluble and not aggregated. The uncleaved monomers can be conveniently purified and used in the method to produce RSV F complexes. Thus, it is preferred that purified RSV F ectodomain polypeptide monomers are used in the method. The RSV F ectodomain polypeptides that are provided and used in the method are preferably uncleaved RSV F ectodomain polypeptides, and more preferably the uncleaved RSV F ectodomain polypeptides contain altered furin cleavage sites. In more particular embodiments, the amino acid sequence of the RSV F ectodomain polypeptides is selected from the group consisting of: SEQ ID NO: 8 (Del21 Furx), SEQ ID NO: 3 (Furmt), SEQ ID NO: 4 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag and/or fusion peptide, is altered or omitted.

The RSV F ectodomain polypeptides (e.g., uncleaved RSV F ectodomain polypeptides) will usually be prepared by expression in a recombinant host system by expression of recombinant constructs that encode the ectodomains in suitable recombinant host cells, although any suitable methods can be used. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B 1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insert cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding RSV F protein ecto-domains can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

RSV F protein ecto-domain polypeptides can be purified using any suitable method. For example, methods for purifying RSV F ecto-domain polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the RSV F protein ecto-domain polypeptides can include a “tag” that facilitates purification, such as an epitope tag or a HIS tag. Such tagged polypeptides can conveniently be purified, for example from conditioned media, by chelating chromatography or affinity chromatography.

Polypeptides may include additional sequences in addition to the RSV F sequences. For example, a polypeptide may include a sequence to facilitate purification (e.g., a poly-His sequence) or a C-terminal 6-helix bundle forming moiety. Similarly, for expression purposes, the natural leader peptide of F protein may be substituted for a different one.

Oligomerization polypeptides contain an oligomerization region and if desired can further contain a functional region as described herein. Suitable amino acid sequences for the oligomerization regions (e.g., the amino acid sequence of the HRA region of RSV F) are well known in the art as are suitable functional regions. The oligomerization polypeptide can be prepared using any suitable method, such as by chemical synthesis, recombinant expression in a suitable host cell, chemical conjugation and the like.

In other aspects, the invention relates to a method for producing a RSV F complex that contains three RSV F ectodomain polypeptides, at least one of which contains a C-terminal 6-helix bundle forming moiety, but does not include an oligomerization polypeptide. The method for producing such complexes is substantially the same as the method for producing complexes that contain an oligomerization polypeptide, but omitting the oligomerization polypeptide. In particular, the method includes a) providing RSV F ectodomain polypeptides that contain a C-terminal 6-helix bundle forming moiety, and b) combining the RSV F ectodomain polypeptides under conditions suitable for the formation of an RSV F complex, whereby a RSV F complex is produced that comprises three RSV F ectodomain polypeptides and is characterized by a six-helix bundle formed by the C-terminal 6-helix bundle forming moiety and the endogenous HRB region.

When RSV F complexes that contain cleaved RSV F ectodomain polypeptides are desired, the optional step c) cleaving the RSV F protein ectodomain polypeptides in the produced complex with a suitable protease can be used. Suitable proteases include any protease that can cleave the RSV F ectodomain polypeptide (preferably an uncleaved RSV F ectodomain polypeptide) to form F1 and F2 subunits. Usually, the protease will cleave a natural or inserted cleavage site between about position 101 to about position 161. One protease that can be used is trypsin. In general, trypsin digestion of the RSV F complex is performed using 1:1000 trypsin:RSV F complex by weight, or 10-15 BAEE units of trypsin for 1 mg of RSV F complex. In a typical reaction, trypsin from bovine plasma (Sigma Aldrich, T8802: 10,000-15,000 BAEE units/mg trypsin) is diluted to a 1 mg/ml concentration in 25 mM Tris pH 7.5, 300 mM NaCl and RSV F protein ecto-domain polypeptide (in 25 mM Tris pH 7.5, 300 mM NaCl) is digested for 1 hour at 37° C. The cleavage reaction can be stopped using a trypsin inhibitor.

In some embodiments, the method comprises a) providing RSV F ectodomain polypeptides and oligomerization polypeptides, and b) combining the RSV F ectodomain polypeptides and at least one oligomerization polypeptide under conditions suitable for the formation of an RSV F complex, whereby a RSV F complex is produced that comprises three of said RSV F ectodomain polypeptides, at least one of said oligomerization polypeptide, and is characterized by a six-helix bundle. The six-helix bundle comprises the HRB region of each RSV F ectodomain polypeptide and the oligomerization domain of each oligomerization peptide. In more specific embodiments, the oligomerization domain of the oligomerization peptide comprises the amino acid sequence of the HRA region of RSV F, and the six-helix bundle comprises the HRB region of each RSV F ectodomain polypeptide and the HRA region of each oligomerization peptide. In a preferred embodiment, three oligomerization domains of the oligomerization peptide comprise the amino acid sequence of the HRA region of RSV F, and the six-helix bundle comprises the HRB region of each of the three RSV F ectodomain polypeptide and the HRA region of each of the three oligomerization peptides.

In other embodiments, the method comprises a) providing recombinant RSV F ectodomain polypeptides that comprises a C-terminal 6-helix bundle forming moiety and oligomerization polypeptides, and b) combining the recombinant RSV F ectodomain polypeptides and oligomerization polypeptides under conditions suitable for the formation of an RSV F complex, whereby a RSV F complex is produced that comprises three of said RSV F ectodomain polypeptides, three of said oligomerization polypeptides, and is characterized by a six-helix bundle. The six-helix bundle comprises the C-terminal 6-helix bundle forming moiety of each recombinant RSV F ectodomain polypeptide and the oligomerization domain of each oligomerization peptide. In more specific embodiments, the C-terminal 6-helix bundle forming moiety is the HRA region of RSV F or HIV gp41, and the oligomerization domain of the oligomerization peptide comprises the amino acid sequence of the HRB region of RSV F or HIV gp41, respectively. In such embodiments, the six-helix bundle comprises the C-terminal 6-helix bundle forming moiety (i.e., the inserted HRA region) of each RSV F ectodomain polypeptide and the HRB region of each oligomerization peptide.

The invention also includes RSV F complexes produced using the methods described herein.

Immunogenic Compositions

The invention provides immunogenic compositions that comprise the RSV F complexes disclosed herein. The compositions are preferably suitable for administration to a mammalian subject, such as a human, and include one or more pharmaceutically acceptable carrier(s) and/or excipient(s), including adjuvants. A thorough discussion of such components is available in Gennaro (2000) Remington: The Science and Practice of Pharmacy. 20th edition, ISBN: 0683306472. Compositions will generally be in aqueous form. When the composition is an immunogenic composition, it will elicit an immune response when administered to a mammal, such as a human. The immunogenic composition can be used to prepare a vaccine formulation for immunizing a mammal.

The immunogenic compositions may include a single active immunogenic agent, or several immunogenic agents. For example, the compositions can contain an RSV F complex and one or more other RSV proteins (e.g., a G protein and/or an M protein) and/or one or more immunogens from other pathogens. The immunogenic composition can comprise a monovalent RSV F complex that contains three RSV F ectodomains and three HRA peptides and if desired can contain one or more additional antigens from RSV F or another pathogen. In one example, the immunogenic composition is divalent and comprises an RSV F complex that also contains another RSV F antigen, such as RSV G protein. As described herein, such multivalent complexes can be produced using an oligomerization polypeptide that contains an oligomerization region that is operably linked to an amino acid sequence from RSV G, such as an amino acid sequence from the central domain of RSV G.

The composition may include preservatives such as thiomersal or 2-phenoxyethanol. It is preferred, however, that the vaccine should be substantially free from (i.e., less than 5 μg/ml) mercurial material, e.g., thiomersal-free. Immunogenic compositions containing no mercury are more preferred. Preservative-free immunogenic compositions are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, and the like.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range. The pH of a composition will generally be between 5.0 and 8.1, and more typically between 6.0 and 8.0, e.g., between 6.5 and 7.5, or between 7.0 and 7.8. A process of the invention may therefore include a step of adjusting the pH of the bulk vaccine prior to packaging.

The composition is preferably sterile. The composition is preferably non-pyrogenic, e.g., containing <1 EU (endotoxin unit, a standard measure) per dose, and preferably <0.1 EU per dose. The composition is preferably gluten free. Human vaccines are typically administered in a dosage volume of about 0.5 ml, although a half dose (i.e., about 0.25 ml) may be administered to children.

Immunogenic compositions of the invention may also comprise one or more immunoregulatory agents. Preferably, one or more of the immunoregulatory agents include one or more adjuvants, for example two, three, four or more adjuvants. The adjuvants may include a TH1 adjuvant and/or a TH2 adjuvant, further discussed below.

Preferably, the immunogenic composition comprises a RSV F complex that displays an epitope present in a pre-fusion conformation of RSV-F glycoprotein. An exemplary composition comprises an RSV F complex that contains cleaved RSV F ecto-domain polypeptides. Another exemplary composition comprises an RSV F complex that contains uncleaved RSV F ecto-domain polypeptides.

Methods of Treatment, and Administration

Compositions of the invention are suitable for administration to mammals, and the invention provides a method of inducing an immune response in a mammal, comprising the step of administering a composition (e.g., an immunogenic composition) of the invention to the mammal. The compositions (e.g., an immunogenic composition) can be used to produce a vaccine formulation for immunizing a mammal. The mammal is typically a human, and the RSV F complex typically contains human RSV F ecto-domain polypeptides. However, the mammal can be any other mammal that is susceptible to infection with RSV, such as a cow that can be infected with bovine RSV.

The invention also provides a composition for use as a medicament, e.g., for use in immunizing a patient against RSV infection.

The invention also provides the use of a RSV F complex as described above in the manufacture of a medicament for raising an immune response in a patient.

The immune response raised by these methods and uses will generally include an antibody response, preferably a protective antibody response. Methods for assessing antibody responses after RSV vaccination are well known in the art.

Compositions of the invention can be administered in a number of suitable ways, such as intramuscular injection (e.g., into the arm or leg), subcutaneous injection, intranasal administration, oral administration, intradermal administration, transcutaneous administration, transdermal administration, and the like. The appropriate route of administration will be dependent upon the age, health and other characteristics of the mammal. A clinician will be able to determine an appropriate route of administration based on these and other factors.

Immunogenic compositions, and vaccine formulations, may be used to treat children and adults, including pregnant women. Thus a subject may be less than 1 year old, 1-5 years old, 5-15 years old, 15-55 years old, or at least 55 years old. Preferred subjects for receiving the vaccines are the elderly (e.g., >50 years old, >60 years old, and preferably >65 years) and pregnant women. The vaccines are not suitable solely for these groups, however, and may be used more generally in a population.

Treatment can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Administration of more than one dose (typically two doses) is particularly useful in immunologically naïve patients. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, and the like.).

Vaccine formulations produced using a composition of the invention may be administered to patients at substantially the same time as (e.g., during the same medical consultation or visit to a healthcare professional or vaccination centre) other vaccines.

### Other Viruses

As well as being used with human RSV, the invention may be used with other members of the Pneumoviridae and Paramyxoviridae, including, but not limited to, bovine respiratory syncytial virus, parainfluenzavirus 1, parainfluenzavirus 2, parainfluenzavirus 3, and parainfluenzavirus 5.

Thus the invention provides an immunogenic composition comprising a F glycoprotein from a Pneumoviridae or Paramyxoviridae, wherein the F glycoprotein is in pre-fusion conformation.

The invention also provides an immunogenic composition comprising a polypeptide that displays an epitope present in a pre-fusion conformation of the F glycoprotein of a Pneumoviridae or Paramyxoviridae, but absent the glycoprotein's post fusion conformation.

The invention also provides these polypeptides and compositions for use in immunization, etc.

### General

The term “comprising” encompasses “including” as well as “consisting” and “consisting essentially of” e.g., a composition “comprising” X may consist exclusively of X or may include something additional e.g., X+Y.

The word “substantially” does not exclude “completely” e.g., a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, it is preferably one that has been approved for use in human vaccine production e.g., as in Ph Eur general chapter 5.2.3.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### EXEMPLIFICATION

The following examples are merely illustrative of the scope of the present invention and therefore are not intended to limit the scope in any way.

### Example 1

### Purification Protocol for RSV F Proteins from Insect Cells

Baculoviruses expressing RSV F constructs were propagated as follows:

One hundred microliters of P1 stock virus were added to 50 mls of SF9 cells (Invitrogen) diluted to 0.8×10⁶/ml (grown in Sf500 media) and allowed to infect/grow for approximately 5-6 days. The infection was monitored using the Cedex instrument. Baculovirus growth was considered complete when cell viability was <50%, while cell diameter predominantly increased from ˜13 nm to ˜16 nm.

One ml of P2 stock was added to 1 liter of Sf9 cells diluted to 0.8×10⁶/ml and was allowed to grow for 5-6 days. The infection was monitored using the Cedex instrument. Baculovirus growth was considered complete when cell viability was <50%, while cell diameter predominantly increased from ˜13 nm to ˜16 nm.

Expression was carried out in cultures of either Sf9 cells or HiFive cells (Invitrogen) in which, unless a test expression was done to determine an appropriate m.o.i., 10 mls of P3 (passage 3) baculovirus stock was added to every liter of cells at 2×10⁶/ml. Expression was allowed to continue for ˜72 hours.

Cells were harvested, after taking an aliquot of cell/media suspension for SDS-PAGE analysis, by pelleting the cells from the media by centrifuging the cells at 3000 r.p.m. for ˜30 minutes.

Copper (II) sulfate was added to the media to a final concentration of 500 micromolar and 1 liter of media with copper was added to ˜15 mls of chelating IMAC resin (BioRad Profinity).

Protein-bound resin was then separated from flow-through using a gravity column. The resin was washed with at least 10 resin volumes of equilibration buffer (25 mM Tris pH 7.5, 300 mM NaCl), and protein was eluted with at least 10 resin volumes of elution buffer (25 mM Tris pH 7.5, 300 mM NaCl, 250 mM imidazole).

The elution solution was spiked with EDTA-free complete protease inhibitor (Pierce) and EDTA to a final concentration of 1 mM. The elution solution was then dialyzed at least twice at 4° C. against 16 volumes of equilibration buffer. The elution solution was loaded onto one or two HiTrap Chelating columns preloaded with Ni⁺⁺. (A single 5 ml column is typically sufficient for 10 liters of expression.) Protein was eluted from the column using an FPLC capable of delivering a gradient of elution buffer with the following gradient profile (2 ml/min flow rate)

a. 0 to 5% Elution buffer over 60 mls

b. 5 to 40% Elution buffer over 120 mls

c. 40 to 100% Elution buffer over 60 mls

Fractions containing RSV F protein were evaluated by SDS-PAGE analysis using Coomassie staining and/or western blotting (typically, RSV F elutes off ˜170 mls into the gradient): the material was concentrated to approximately 0.5-1 mg/ml; and EDTA was added to 1 mM final concentration

Using an FPLC, 1 ml fractions were collected. The RSV F material (retention volume approximately 75 ml) was resolved from the insect protein contaminates (retention time approximately 60 ml) by size exclusion chromatography (SEC) with a 16/60 Superdex column (GE Healthcare) using with equilibration buffer as the mobile phase.

Fractions were analyzed using SDS-PAGE with Coomassie staining and sufficiently pure RSV F material was pooled and concentrated to approximately 1 mg/ml.

### Example 2

### Design of RSV F Uncleavable Monomer+HRA Peptide

HRA peptide (the oligomerization polypeptide) synthesized by Anaspec (RSV F HRA peptide, RSV residues 160-207) was resuspended into SEC buffer (25 mM Tris pH 7.5, 300 mM NaCl) and UV absorbance at 280 nm (1 AU per 1 mg/ml: estimated) was used to estimate protein concentration.

RSV F uncleavable ectodomain (Delp21Furx, the ectodomain polypeptide) was purified according to the RSV F insect purification protocol described in Example 1. The ectodomain was purified by SEC preparatory purification at an elution volume of approximately 75 ml, consistent with the ectodomain being monomeric. An ˜0.75 mg/ml (estimated by UV as above) solution was used for complex formation.

Next, 0.5 mls of ˜0.75 mg/ml RSV F monomer was added to 0.5 mls HRA peptide solution, and 1 ml of the complex solution was separated on a SEC column according to the RSV F purification protocol. The result is summarized in Table 1.

TABLE 1
SEC retention volume of RSV F monomer
with or without addition of HRA peptide

| | Retention Volume |
|---|---|
| Species | Superdex P200 (ml) |
| RSV Monomer (Delp23 Furdel) | ~75 mls |
| RSV Trimer (FP deletion) | ~65 mls |
| RSV Monomer + HRA peptide | ~60 mls |

Table 1 shows the change in retention volume of the RSV F monomer (Delp23 Furdel) upon addition of HRA peptides. The uncleaved monomer alone runs with a retention time of ˜75 mls, while the monomer with added HRA peptides runs with a retention volume of ˜60 mls. For comparison, the published RSV F trimer (fusion peptide deletion) runs with a retention volume of ˜65 mls. The retention volume for the RSV F monomer+HRA sample was ˜60 mls, more consistent with a trimer elution than a monomer. This shift in retention volume suggests peptide-F protein interaction and formation of a trimer of complexes between the HRA peptides and the RSV F uncleavable ectodomains (that is, a hetero-hexamer with three HRA peptides and three F uncleavable ectodomains).

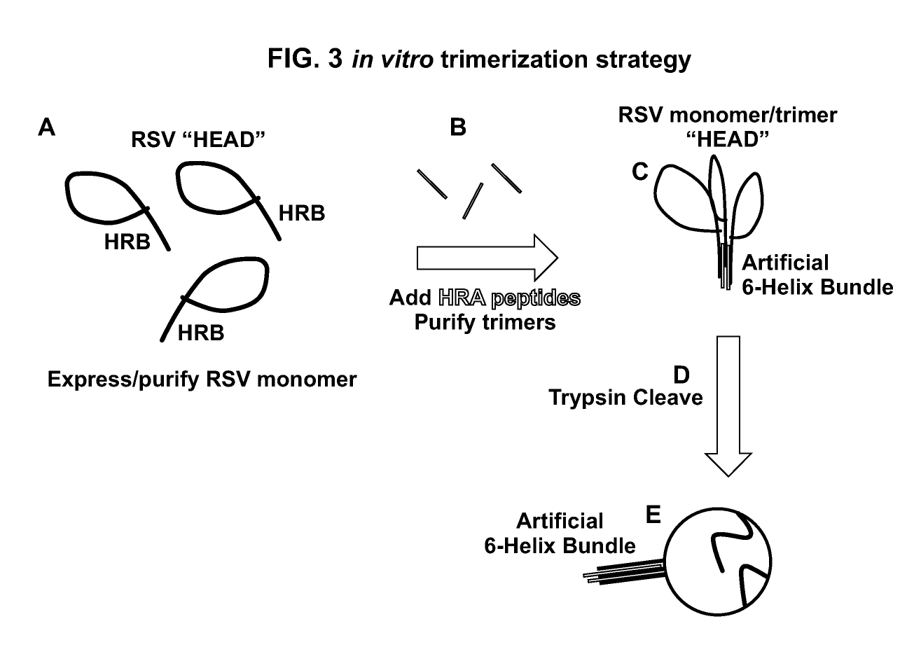

This uncleavable ectodomain F:HRA peptide complex will be evaluated by electron microscopy (EM) to determine if a three-lobed species or a prefusion globular head is formed (as predicted in FIG. 3). Additionally, the peptide complex formation will be repeated with cleavable RSV F ectodomain that can be trypsin digested to F1/F2 species. If the prefusion F globular head is formed, and this prefusion RSV F behaves similarly to parainfluenza F, we expect that stabilizing the prefusion form will prevent rosette formation.

### Example 3

### Addition of a C-Terminal 6-Helix Bundle Forming Sequence

A sequence, such as an additional RSV HRA or HIV gp41 HRA is added to the complex described in Example 2 to form a C-terminal 6-helix bundle, thus permitting trimerization with addition of RSV HRB or HIV gp41 HRB, respectively. This may have an additional advantage of constraining RSV HRB from the monomer into its native prefusion HRB trimer stalk, instead of the postfusion-like 6-helix bundle.

### Example 4

### Addition of HRB Disulfides

HRB disulfides are added to the HRB described in Example 2. Thus, when trimerization of the monomer occurs, the cysteine additions are in appropriate positions to form the desired disulfides, providing an additional level of prefusion stability.

### Example 5

### Addition of Conjugated Proteins Fused to Peptides

Instead of adding HRA, HRB or gp41 peptides (Example 3), conjugated proteins fused with these peptides are added, such as RSV G, albumin or KLF conjugate protein. For example, an HRA peptide-RSV G central domain construct is added to the F monomer protein. Upon trimerization induced by the HRA peptide, the RSV G central domain protein is bound to F making an F/G complex, which may provide further immunogenicity upon vaccination.

The entire teachings of all documents cited herein are hereby incorporated herein by reference.

What is claimed is:

1. A respiratory syncytial virus F (RSV F) complex, comprising
three RSV F ectodomain polypeptides each comprising an endogenous HRA region, and
at least one oligomerization polypeptide, wherein the three ectodomain polypeptides and the at least one oligomerization polypeptide form a six-helix bundle, with the proviso that the endogenous HRA regions of the RSV F polypeptides are not part of the six-helix bundle.

2. The RSV F complex of claim 1, wherein:
(i) each RSV F ectodomain polypeptide comprises an HRB region and each oligomerization polypeptide comprises an oligomerization region; and/or
(ii) the six helix bundle comprises the HRB region of each RSV F ectodomain polypeptide and the oligomerization region of each oligomerization peptide.

3. The RSV F complex of claim 1, wherein each oligomerization region comprises an RSV F HRA amino acid sequence.

4. The RSV F complex of claim 1, wherein the complex consists of the three RSV F ectodomain polypeptides and three oligomerization polypeptides.

5. The RSV F complex of claim 1, wherein one or more of said oligomerization polypeptides further comprises a functional region that is operably linked to the oligomerization region.

6. The RSV F complex of claim 5, wherein the functional regions are independently selected from the group consisting of an immunogenic carrier protein, an antigen, a particle-forming polypeptide, a lipid, and polypeptides that can associate the oligomerization polypeptide with a liposome or particle.

7. The RSV F complex of claim 6, wherein the functional region is an antigen, and wherein the antigen is RSV G.

8. The RSV F complex of claim 1, wherein:
(i) one or more of the RSV F ectodomain polypeptides is an uncleaved RSV F ectodomain polypeptide;
(ii) one or more of the RSV F ectodomain polypeptides is a cleaved RSV F ectodomain polypeptide; and/or
(iii) each of the RSV F ectodomain polypeptides contain one or more altered furin cleavage sites.

9. The RSV F complex of claim 1, wherein the amino acid sequence of the RSV F ectodomain polypeptides corresponding to residues 100-150 of the wild type RSV F polypeptide is selected from the group consisting of: SEQ ID NO: 8 (Del21 Furx), SEQ ID NO: 3 (Furmt), SEQ ID NO: 4 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag and/or fusion peptide, is omitted or altered.

10. The RSV F complex of claim 1, wherein at least one of the RSV F ectodomain polypeptides is a recombinant polypeptide that comprises a C-terminal 6-helix bundle forming moiety.

11. The RSV F complex of claim 10, wherein the C-terminal six-helix bundle forming moiety comprises a heptad repeat region of the fusion protein of an enveloped virus.

12. The RSV F complex of claim 11, wherein the heptad repeat region is selected from the group consisting of RSV F HRA, RSV HRB, and HIV gp41 HRA.

13. The RSV F complex of claim 10, wherein the six-helix bundle comprises the C-terminal 6-helix bundle forming moiety of three recombinant RSV F ectodomain polypeptides and the oligomerization region of each oligomerization peptide.

14. The RSV F complex of claim 1, wherein:
(i) the RSV F ectodomain polypeptides are in the pre-fusion conformation;
(ii) the complex is characterized by a rounded shape when viewed in negatively stained electron micrographs; and/or
(iii) the complex comprises pre-fusion epitopes that are not present on post-fusion forms of RSV F.

15. A respiratory syncytial virus F (RSV F) complex, comprising three RSV F ectodomain polypeptides that each contain an endogenous HRA region and an endogenous HRB region, at least one of said RSV F ectodomain polypeptides further comprising a C-terminal 6-helix bundle forming moiety, wherein the complex is characterized by a six-helix bundle formed by the C-terminal 6-helix bundle forming moiety and the endogenous HRB region.

16. A method for producing a respiratory syncytial virus F (RSV F) complex, comprising:
a) providing RSV F protein ectodomain polypeptides and at least one oligomerization polypeptide, and
b) combining the RSV F ectodomain polypeptides and the at least one oligomerization polypeptide under conditions suitable for the formation of a RSV F complex, whereby a RSV F complex is produced in which three of said RSV F ectodomain polypeptides and at least one of said oligomerization polypeptide form a six-helix bundle, with the proviso that the endogenous HRA regions of the RSV F ectodomain polypeptides are not part of the six-helix bundle.

17. The method of claim 16, wherein the RSV F ectodomain polypeptides provided in a):
(i) are uncleaved RSV F ectodomain polypeptides;
(ii) each contain one or more altered furin cleavage sites;
(iii) are purified monomers; and/or
(iv) are expressed in insect cells, mammalian cells, avian cells, yeast cells, Tetrahymena cells or combinations thereof.

18. The method of claim 16, further comprising c) cleaving the RSV F protein ectodomain polypeptides in the produced complex with a protease.

19. The method of claim 16, wherein each RSV F ectodomain polypeptide comprises an HRB region and each oligomerization polypeptide comprises an oligomerization region.

20. The method of claim 19, wherein the six-helix bundle comprises the HRB region of each RSV F ectodomain polypeptide and the oligomerization region of each oligomerization peptide.

21. The method of claim 16, wherein:
(i) the at least one oligomerization polypeptide comprises an RSV F HRA amino acid sequence;
(ii) the complex consists of the three RSV F ectodomain polypeptides and three oligomerization polypeptides;
(iii) one or more of said oligomerization polypeptides further comprise a functional region that is operably linked to the oligomerization region;
(iv) the amino acid sequence of the RSV F ectodomain polypeptides provided in step a) corresponding to residues 100-150 of the wild type RSV F polypeptide is selected from the group consisting of: SEQ ID NO: 8 (Del21 Furx), SEQ ID NO: 3 (Furmt), SEQ ID NO: 3 (Furdel), SEQ ID NO: 5 (Furx), SEQ ID NO: 6 (Furx R113Q, K123N, K124N), SEQ ID NO: 7 (Furx R113Q, K123Q, K124Q), SEQ ID NO: 9 (Delp23Furx), SEQ ID NO: 10 (Delp21 furdel), SEQ ID NO: 11 (Delp23 furdel), and any of the foregoing in which the signal peptide and/or HIS tag and/or fusion peptide, is omitted or altered; and/or
(v) at least one of the RSV F ectodomain polypeptides is a recombinant polypeptide that comprises a C-terminal 6-helix bundle forming moiety.

22. The method of claim 21, wherein the C-terminal 6-helix bundle forming moiety comprises a heptad repeat region of the fusion protein of an enveloped virus.

23. The method of claim 22, wherein the heptad repeat region is selected from the group consisting of RSV F HRA, RSV F HRB, and HIV gp41 HRA.

24. The method of claim 20, wherein the six-helix bundle comprises the C-terminal 6-helix bundle forming moiety of three recombinant RSV F ectodomain polypeptides and the oligomerization region of each oligomerization peptide.

25. The method of claim 16, wherein the RSV F ectodomain polypeptides in the complex that is produced:
(i) are in the pre-fusion conformation;
(ii) are characterized by a rounded shape when viewed in negatively stained electron micrographs; and/or
(iii) comprise prefusion epitopes that are not present on post-fusion forms of RSV F.

26. A method for producing a respiratory syncytial virus F (RSV F) complex, comprising:
a) providing RSV F protein ectodomain polypeptides that contain a C-terminal 6-helix bundle forming moiety, and
b) combining the RSV F ectodomain polypeptides under conditions suitable for the formation of a RSV F complex, whereby a RSV F complex is produced that comprises three RSV F ectodomain polypeptides and is characterized by a six-helix bundle formed by the C-terminal 6-helix bundle forming moiety and the endogenous HRB region.

27. A respiratory syncytial virus F (RSV F) complex produced by the method of claim 16.

28. An immunogenic composition comprising a respiratory syncytial virus F (RSV F) complex according to claim 1 or 27.

29. A method of inducing an immune response to RSV F in a subject comprising administering an immunogenic composition of claim 28 to the subject.


SEQUENCE LISTING (40 sequences)

SEQ ID NO: 1 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 574 aa, Respiratory syncytial virus
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 3 - protein, 51 aa, Respiratory syncytial virus
TPATNNRARKELPRFMNYTLNNAKKTNVTLSKKRKKKFLGFLLGVGSAIAS

SEQ ID NO: 4 - protein, 48 aa, Respiratory syncytial virus
TPATNNRARQELPRFMNYTLNNAKKTNVTLSKKRFLGFLLGVGSAIAS

SEQ ID NO: 5 - protein, 51 aa, Respiratory syncytial virus
TPATNNQAQNELPRFMNYTLNNAKKTNVTLSQNQNQNFLGFLLGVGSAIAS

SEQ ID NO: 6 - protein, 51 aa, Respiratory syncytial virus
TPATNNQAQNELPQFMNYTLNNANNTNVTLSQNQNQNFLGFLLGVGSAIAS

SEQ ID NO: 7 - protein, 51 aa, Respiratory syncytial virus
TPATNNQAQNELPQFMNYTLNNAQQTNVTLSQNQNQNFLGFLLGVGSAIAS

SEQ ID NO: 8 - protein, 30 aa, Respiratory syncytial virus
TPATNNQAQNQNQNQNFLGFLLGVGSAIAS

SEQ ID NO: 9 - protein, 28 aa, Respiratory syncytial virus
TPATNNQAQNQNQNFLGFLLGVGSAIAS

SEQ ID NO: 10 - protein, 30 aa, Respiratory syncytial virus
TPATNNRARQQNQQQRFLGFLLGVGSAIAS

SEQ ID NO: 11 - protein, 28 aa, Respiratory syncytial virus
TPATNNRARQQQQRFLGFLLGVGSAIAS

SEQ ID NO: 12 - protein, 51 aa, Respiratory syncytial virus
TPATNNRARRELPQFMNYTLNNAQQTNVTLSQNQNQNFLGFLLGVGSAIAS

SEQ ID NO: 13 - protein, 51 aa, Respiratory syncytial virus
TPATNNQAQNELPQFMNYTLNNAQQTNVTLSKKRKRRFLGFLLGVGSAIAS

SEQ ID NO: 14 - protein, 51 aa, Respiratory syncytial virus
TPATNNIEGRELPRFMNYTLNNAKKTNVTLSKKIEGRFLGFLLGVGSAIAS

SEQ ID NO: 15 - protein, 41 aa, Respiratory syncytial virus
TPPTNNRARRELPRFMNYTLNNAKKTNVTLSKKRKRRAIAS

SEQ ID NO: 16 - protein, 300 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGLEGEVNKIKSALLSTNKAVVSLSNGVSVLTSKVLDLKNYIDKQLLPIV

SEQ ID NO: 17 - protein, 290 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGLEGEVNKIKSALLSTNKAVVSLSNGVSVLTSKVLDLKN

SEQ ID NO: 18 - protein, 337 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGRLKNPPKKPKDDYHFEVFNFVPCSICGNNQLCKSICKTIPGGSAGSGLEGEVNKIKSALLSTNKAVVSLSNGVSVLTSKVLDLKN

SEQ ID NO: 19 - protein, 337 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGRQNKPPSKPNNDFHFEVFNFVPCSICSNNPTCWAICKRIPGGSAGSGLEGEVNKIKSALLSTNKAVVSLSNGVSVLTSKVLDLKN

SEQ ID NO: 20 - protein, 333 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGRLKNPPKKPKDDYHFEVFNFVPCSICGNNQLCKSICKTIPGGSAGSGPSDEFDASISQVNEKINQSLAFIRKSDELLHNVN

SEQ ID NO: 21 - protein, 333 aa, Artificial Sequence
  Description of Artificial Sequence Synthetic polypeptide
MHHHHHHGSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGSLEVLFQGPGGSAGSGRQNKPPSKPNNDFHFEVFNFVPCSICSNNPTCWAICKRIPGGSAGSGPSDEFDASISQVNEKINQSLAFIRKSDELLHNVN

SEQ ID NO: 22 - protein, 543 aa, Artificial Sequence
  Description of Artificial Sequence Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNLEGEVNKIKSALLSTNKAVVSLSNGVSVLTSKVLDLKNGGSAGSGHHHHHH

SEQ ID NO: 23 - protein, 543 aa, Artificial Sequence
  Description of Artificial Sequence Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLCFPSDEFCASISQVNEKINQSLAFIRKCCELLHNLEGEVNKIKSALLSTNKAVVSLSNGVSVLTSKVLDLKNGGSAGSGHHHHHH

SEQ ID NO: 24 - protein, 574 aa, Artificial Sequence
  Description of Artificial Sequence Synthetic consensus polypeptide
MELLILKANAITTILAAVTFCFASSQNITEEFYQSTCSAVSKGYLSALRTGWYTSVITIELSNIKENKCNGTDAKVKLIKQELDKYKNAVTELQLLMQSTPAANNRARRELPRXMNYTLNNTKKTNVTLSKKRKRRFLGFLLGVGSAIASGXAVSKVLHLEGEVNKIKSALLSTNKAVVSLSNGVSVLTSKVLDLKNYIXKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAXTCKVQSNRVFCDTMNSLTLPSEVNLCNVDIFNPKYDCKIMTSKTDXSSSVITSLGAIVSCYGKTKCTASNKNRGIIKTFSNGCDYCSNKGVDTVSVGNTLYYVNKQEGKSLYVKGEPIINXYDPLVFPSDEFDASISQVNEKINQSLAFIRKSDELLHNVNAGKSTTNIMITTIIIVIIVXLLSLIAXGLLLYCKARSTPVTLSKDQLSGINNIAFSN

SEQ ID NO: 25 - protein, 51 aa, Respiratory syncytial virus
TPATNNRARRELPRFMNYTLNNAKKTNVTLSKKRKRRFLGFLLGVGSAIAS

SEQ ID NO: 26 - protein, 42 aa, Respiratory syncytial virus
TPATNNRARRELPRFMNYTLNNAKKTNVTLSKKRKRRSAIAS

SEQ ID NO: 27 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 28 - protein, 574 aa, Respiratory syncytial virus
MELLIHRLSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIGTVIEFQQKNSRLLEINREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 29 - protein, 574 aa, Respiratory syncytial virus
MELLIHRLSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEINREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 30 - protein, 574 aa, Respiratory syncytial virus
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 31 - protein, 574 aa, Respiratory syncytial virus; identical to SEQ ID NO: 2

SEQ ID NO: 32 - protein, 4 aa, Respiratory syncytial virus
RARK

SEQ ID NO: 33 - protein, 4 aa, Respiratory syncytial virus
RARQ

SEQ ID NO: 34 - protein, 4 aa, Respiratory syncytial virus
QAQN

SEQ ID NO: 35 - protein, 4 aa, Respiratory syncytial virus
IEGR

SEQ ID NO: 36 - protein, 4 aa, Respiratory syncytial virus
RKKK

SEQ ID NO: 37 - protein, 4 aa, Respiratory syncytial virus
QNQN

SEQ ID NO: 38 - protein, 4 aa, Respiratory syncytial virus
QQQR

SEQ ID NO: 39 - protein, 4 aa, Respiratory syncytial virus
IEGR

SEQ ID NO: 40 - protein, 6 aa, Artificial Sequence
  Description of Artificial Sequence Synthetic 6xHis tag
HHHHHH